(19) United States
(12) Patent Application Publication
(10) Pub. No.: US 2026/0356779 A1
(43) Pub. Date: Nov. 12, 2026

(54) COMPOSITIONS INCLUDING 1,1,2-TRIFLUOROETHANE (HFC-143) AND METHODS FOR PRODUCING trans-1,2-DIFLUOROETHYLENE (HFO-1132E)

(71) Applicant: Solstice Advanced Materials US, Inc., Morris Plains, NJ (US)

(72) Inventors: Haiyou Wang, Amherst, NY (US); Terris Yang, East Amherst, NY (US); Akbar Mahdavi-Shakib, Buffalo, NY (US); Selma Bektesevic, Williamsville, NY (US); Yian Zhai, Lockport, NY (US); Dimitrios Papanastasiou, Buffalo, NY (US); Haluk Kopkalli, Staten Island, NY (US)

(21) Appl. No.: 19/668,530

(22) Filed: May 5, 2026

Related U.S. Application Data
(60) Provisional application No. 63/803,500, filed on May 9, 2025.
(60) Provisional application No. 63/852,172, filed on Jul. 28, 2025.

(51) Int. Cl. C07C 21/18; C07C 17/25; C07C 17/383

(52) U.S. Cl. CPC C07C 21/18; C07C 17/25; C07C 17/383

(57) ABSTRACT

A reactant composition including 1,1,2-trifluoroethane (HFC-143) is dehydrofluorinated in a liquid phase reactor to produce a product composition including Cis-1,2-difluoroethylene (HFO-1132(Z)). The product composition may be further post-processed to remove impurities and create a final product stream of high purity HFO-1132(Z). The post-processing may also include recycling HFC-143.

### CROSS-REFERENCE TO RELATED APPLICATION

This application claims priority to U.S. Provisional Application No. 63/803,500 filed May 9, 2025, and to U.S. Provisional Application No. 63/852,172 filed Jul. 28, 2025, both of which are herein incorporated by reference in their entirety.

### FIELD

The present disclosure relates generally to a method for producing trans-1,2-difluoroethylene (HFO-1132E) from 1,1,2-trifluoroethane (HFC-143), and more specifically to methods for improving compositions including HFC-143 useful for producing trans-1,2-difluoroethylene (HFO-1132E), for example by managing the formation of and/or converting intermediates in such HFC-143 compositions.

### BACKGROUND

1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Certain intermediates and/or byproducts are produced in the process for manufacturing HFO-1132. The formation and management of such intermediates and byproducts, though inevitable, present challenges, and it would be desirable to convert any useful intermediates into desired products and/or minimize formation of any undesired byproducts in the process to produce the desired product HFO-1132.

### SUMMARY

HFO-1132 and, in particular, HFO-1132E, is produced from 1,1,2-trifluoroethane (HFC-143). HFC-143 may be obtained from a variety of ways, including but not limited to hydrogenating 1,1,2-trichloro-1,2,2-trifluoroethane (CFC-113), chlorotrifluoroethylene (CTFE or CFC-1113) and/or trifluoroethylene (TrFE or HFO-1123). The hydrogenation reaction producing HFC-143 may also produce several intermediates including one or more of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) as well as undesired byproducts including but not limited to HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

The presence of these intermediates (e.g., HCFC-133b, HCFC-133, and/or HCFC-123a) and byproducts (e.g., HCFC-142 isomers) in the feed along with HFC-143 leads to a drop in selectivity to HFO-1132 (Z/E) due to formation of heavies (dimers, trimers, or oligomers that remain in the liquid phase due to their high boiling point). However, these intermediates and byproducts are difficult to separate from HFC-143. In addition, even after challenging separation, the hydrogenation and conversion of the intermediates (e.g., HCFC-133b, HCFC-133, and/or HCFC-123a) to HFC-143 is difficult due to their low reactivity. The present disclosure overcomes separation difficulties by converting HCFC-133 isomers to more easily separable HFO-1123 and HCFC-123a to more easily separable CTFE. HFO-1123 and CTFE are easier to hydrogenate compared to HCFC-133 isomers and HCFC-123a, removing the need for an additional recycle reactor or harsher conditions that are typically needed in a hydrogenation reactor for producing HFC-143 from other starting materials.

In a first step of the present process, a feed including HFC-143 and one or more of the intermediates (e.g., HCFC-133b, HCFC-133, and/or HCFC-123a) is reacted with a base where the one or more intermediates are dehydrochlorinated to increase the relative amount of HFC-143 in the composition by converting such intermediates to other components (e.g., CTFE, HFO-1123) which may be more easily separated from the product composition. Separating the other components from the product composition provides a second composition including higher purity HFC-143. The second composition containing higher purity HFC-143 is then dehydrofluorinated to produce trans-1,2-difluoroethylene (HFO-1132E) and/or cis-1,2-difluoroethylene (HFO-1132Z).

The HFO-1132Z is then isomerized to produce HFO-1132E. More particularly, the HFO-1132Z is formed as a composition with a high amount of HFO-1132Z in which the presence of identified byproducts is controlled and minimized to result in a final HFO-1132E product composition. Due to the controlled and minimized presence of the identified byproducts in the HFO-1132Z composition, the resulting HFO-1132E product composition is substantially free, or entirely free, of other, further byproducts that may be formed from the identified byproducts in the isomerization reaction, and which have been found to be difficult to separate from the HFO-1132E product composition.

In one form thereof, a method for reducing impurities in a composition of 1,1,2-trifluoroethane (HFC-143), comprises: providing a first reactant composition comprising HFC-143 and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a); and reacting the first reactant composition with a base in a first liquid phase reactor to produce a first product composition comprising from 70 mol % to 99.8 mol % of 1,1,2-trifluoroethane (HFC-143), based on total moles of organic components of the first product composition.

In another form thereof, the method for reducing impurities in a composition of 1,1,2-trifluoroethane (HFC-143), comprises: providing a first reactant composition comprising HFC-143 and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a); reacting the first reactant composition with a base in a first liquid phase reactor to produce a first product composition comprising from 70 mol % to 99.8 mol % of 1,1,2-trifluoroethane (HFC-143), based on total moles of organic components of the first product composition; distilling the first product composition to produce a distilled composition comprising at least 90 mol % HFC-143; and further reacting the distilled composition with a second base in a second liquid phase reactor to produce a second product composition; wherein the second reacting step is conducted at a temperature higher than a temperature of the first reacting step.

In yet another form thereof, a composition is provided including: cis-1,2-difluoroethylene (HFO-1132(Z)) in an amount of at least 97 wt. %; 1,1,1-trifluoroethane (HFC-143a) and trifluoroethylene (HFO-1123) together present in a combined amount of greater than 0 ppm and less than 2000 ppm; 1,1,2-trifluoroethane (HFC-143) in an amount greater than 0.01 wt. % and less than 0.75 wt. %; and 1,1-difluoroethane (HFC-152a) in an amount greater than 0.01 wt. % and less than 0.75 wt. %. All amounts are based on a total weight of organic components of the composition.

In a further form thereof, a method for producing HFO-1132(E) is provided. The method including reacting a reactant composition at a temperature of 490 °C, a pressure of 5 psig to 30 psig, and a residence time of 6 seconds to 25 seconds. The reactant composition includes: cis-1,2-difluoroethylene (HFO-1132(Z)) in an amount of at least 97 wt. %; 1,1,1-trifluoroethane (HFC-143a) and trifluoroethylene (HFO-1123) together present in a combined amount of greater than 0 ppm and less than 2000 ppm; 1,1,2-trifluoroethane (HFC-143) in an amount greater than 0.01 wt. % and less than 0.75 wt. %; and 1,1-difluoroethane (HFC-152a) in an amount greater than 0.01 wt. % and less than 0.75 wt. %. All amounts are based on a total weight of organic components of the reactant composition, to produce a crude product composition.

### BRIEF DESCRIPTION OF THE DRAWINGS

FIG. 1 is a schematic diagram of a two-reactor layout for the conversion of 1,1,2-trichloro-1,2,2-trifluoroethane (HFC-143) to 1,2-difluoroethylene (HFO-1132).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

In the Examples below, the designation “R” may be used in connection with the various fluorine-containing molecules described herein, for example, “R-143” refers to 1,1,2-trifluoroethane (HFC-143).

As used herein, the phrase “intermediates” include one or more of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a).

As used herein, the phrase “undesired byproducts” include one or more of 1,1,1-trifluoroethane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

As used herein, the phrase “impurities” includes the intermediates and undesired byproducts as defined above, and any other organic components in a composition of HFC-143 other than HFC-143.

As used herein, the phrase “based on total moles of organic components of the composition” refers only to carbon-containing components and does not include or encompass non-carbon-containing components including but not limited to hydrogen (H₂) or hydrogen chloride (HCl).

As used herein, a “continuous reaction” refers to performing a chemical reaction where at least one of the reactants is provided in a continuous flowing stream.

As used herein, a “batch reaction” refers to a chemical process conducted in a closed system where reactants are added, mixed, and allowed to react for a specific time before the products are removed.

As used herein, a “semi-continuous reaction” refers to a process where reactants are continuously added to a reaction vessel, while the product is removed intermittently or removed continuously.

As used herein, conversion of a reactant molecule (molecule A) during a reaction (molecule A→molecule B) is calculated using the following equation:

$$Molecule A conversion = \frac{Moles A in the feed - moles A in the product}{Moles A in the feed} \times 100 \%$$

As used herein, selectivity to a molecule (molecule B) formed during a reaction (molecule A→molecule B) is calculated using the following equation:

$$Molecule B selectivity = \frac{Moles B in the product}{Moles A in the feed - moles A in the product} \times 100 \%$$

### II. Overview

The present disclosure provides a method for producing E-1,2-difluoroethylene (HFO-1132E) from 1,1,2-trifluoroethane (HFC-143) according to a three-step process shown below (“Process 1”), which includes the following three steps: (i) dehydrochlorinating one or more intermediates and/or undesired byproducts in a feed containing HFC-143 to other components (e.g., CTFE, HFO-1123) which may be more easily separated from the product composition, thus producing a composition with higher purity HFC-143, (ii) dehydrofluorinating HFC-143 to produce a mixture of trans-1,2-difluoroethylene (HFO-1132E) and cis-1,2-difluoroethylene (HFO-1132Z), and (iii) optionally isomerizing HFO-1132Z to HFO-1132E.

Schematic equations for the three steps of Process 1 are represented below:

### Process 1

(i)-(a) CClF₂—CH₂F (HCFC-133b)+M(OH)ₙ→CF₂═CHF (HFO-1123)+M(Cl)ₙ+H₂O (M=K, Na, or Ca; n=1, or 2)
(i)-(b) CHF₂—CHClF (HCFC-133)+M(OH)ₙ→CF₂═CHF (HFO-1123)+MClₙ+H₂O (M=K, Na, or Ca; n=1, or 2)
(i)-(c) CClF₂—CHClF (HCFC-123a)+M(OH)ₙ→CF₂═CClF (CTFE)+MClₙ+H₂O (M=K, Na, or Ca; n=1, or 2)
(i)-(d) CHF₂—CH₂Cl (HCFC-142)+M(OH)ₙ→CF₂═CH₂ (VDF or HFO-1132a)+MClₙ+H₂O (M=K, Na, or Ca; n=1, or 2)
(i)-(e) CClF₂—CH₃ (HCFC-142b)+M(OH)ₙ→CF₂═CH₂ (VDF or HFO-1132a)+MClₙ+H₂O (M=K, Na, or Ca; n=1, or 2)
(ii) CFH₂—CF₂H (HFC-143)+MOHₙ→trans-CFH═CHF (HFO-1132E)+cis-CFH═CFH (HFO-1132Z)+MFₙ+H₂O (M=K, Na, or Ca; n=1, or 2)
(iii) cis-CFH═CFH (HFO-1132Z)→trans-CFH═CHF (HFO-1132E)

HFO-1123 and CTFE produced in Step (i) may be recycled and hydrogenated to produce HFC-143 under the below reactions:
CF₂═CClF (CTFE)+2H₂→CHF₂→CH₂F (HFC-143)+HCl
CF₂═CHF (HFO-1123)+H₂→CHF₂→CH₂F (HFC-143)

While some of the HFC-143 may be dehydrofluorinated during Step (i), the reaction conditions are selectively tailored such that the level of intermediates and undesired byproducts are reduced in the composition with minimal conversion of HFC-143. Without wishing to be bound by theory, the reaction conditions may be tailored because the dehydrofluorination reaction of HFC-143 requires harsher conditions compared to the dehydrochlorination reactions of the intermediates and/or undesired byproducts.

Further, it has been found that selectivity to the target HFO-1132 (Z/E) product is dependent on the HFC-143 purity, and presence of HCFCs (e.g., HCFC-133 isomers and HCFC-123a) in the feed leads to a drop in selectivity to HFO-1132 (Z/E) due to formation of heavies (dimers, trimers, or oligomers that remain in the liquid phase due to their high boiling point).

Further details regarding each of Steps (i), (ii), (iii) are set forth below.

### III. Process Flow

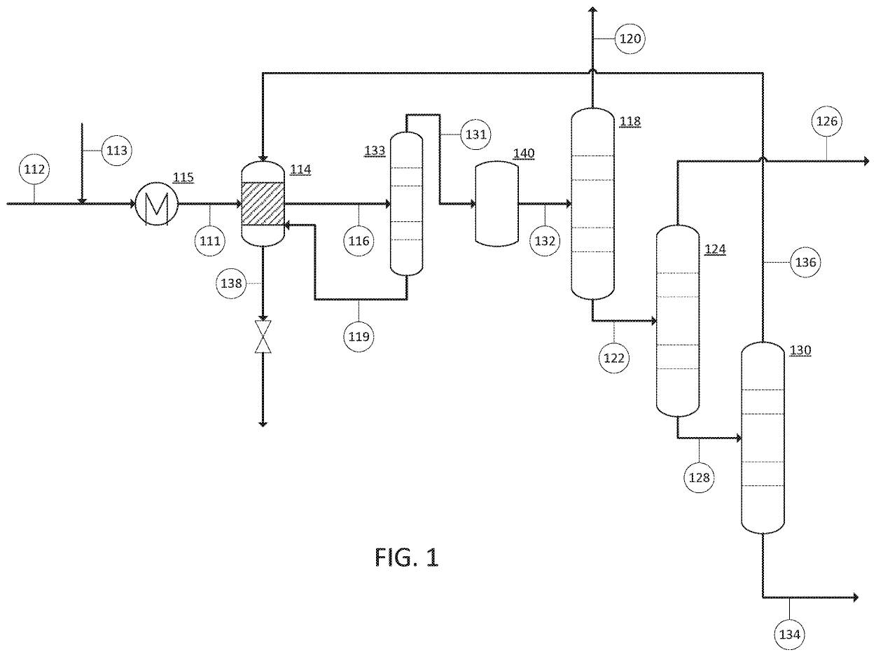

FIG. 1 includes a schematic process flow diagram illustrating suitable components for the reactions in Process 1 and Process 2 for the conversion of 1,1,2-trichloro-1,2,2-trifluoroethane (HFC-143) to 1,2-difluoroethylene (HFO-1132).

A feed stream comprising HFC-143 and one or more intermediates such as HCFC-133b, HCFC-133, and/or HCFC-123a, and/or undesired byproducts including but not limited to HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be sent to a pretreatment reactor (unillustrated). The feed stream may be obtained from a variety of ways, including but not limited to hydrogenating 1,1,2-trichloro-1,2,2-trifluoroethane (CFC-113), chlorotrifluoroethylene (CTFE or CFC-1113) and/or trifluoroethylene (TrFE or HFO-1123).

Inside the pretreatment reactor, the one or more intermediates and undesired byproducts may undergo the dehydrochlorination reaction of Step (i) to produce a first product composition comprising a higher percentage of HFC-143 compared to the feed stream entering the pretreatment reactor. After undergoing pretreatment, the pretreated stream may be sent to a pretreatment distillation column to remove lights (e.g., CTFE, HFO-1123, HFO-1132Z, and HFC-152a) from the pretreated stream. Alternatively, or additionally, a portion of the pretreated stream may be first sent to a separator for removal of unreacted dehydrohalogenating agent, as described below, and then enter the preconditioning distillation column. The top stream from the pretreatment distillation column may be further recycled or sent to a subsequent reactor for further processing. The bottom stream of the preconditioning distillation column contains high purity of HFC-143.

In some cases, the bottom stream from the pretreatment distillation column may be used as feed stream 112. In other cases, feed stream 112 may be a feed stream that comprises relatively high purity of HFC-143 with one or more minor impurities, such as HCFC-133b and/or HCFC-142b, obtained from step (i) without undergoing pretreatment.

Feed stream may be preheated by preheater 115 to heat the stream 112 before entering second reactor 114. The reactor 114 may comprise at least one of a heating jacket and an internal heating coil to heat the reactants. Feed stream 112 may be mixed with a base feed stream 113 before entering second reactor 114, or the base feed stream 113 may be fed directly into the reactor 114 separately from stream 112 (unillustrated). The HFC-143 of feed stream 112 undergoes the dehydrofluorination reaction of Step (ii) in reactor 114 to produce a product stream 116 including a product composition comprising HFO-1132Z and HFO-1132E.

Product stream 116 including the product composition enters a reaction distillation column 133, which initially separates unreacted HFC-143 from the remaining components of the product composition, where a proportion of the unreacted HFC-143 as well as water, is recycled back into reactor 114. During operation, reactor 114 may operate in a batch, semicontinuous, or continuous operation, whereas reactor distillation column may be operated in conjunction with reactor 114 to continuously recirculate unreacted HFC-143 and water into reactor 114. The reactor 114 may comprise a plurality of reactors in parallel or in series. In examples where multiple reactors are used, the product stream from each reactor may be fed to individual distillation columns, the same reaction distillation column 133, and/or only the product stream from the final reactor in a series of reactors may be sent to the reaction distillation column 133. Top stream 131 from reactor distillation column 133 is sent through one or more preconditioning components, such as a dryer 140, which removes excess water from stream 131. Dried product stream 131 enters a first distillation column 118, which is operated to remove the light components in top stream 120 of the product composition generated in the step (ii) reaction. Alternatively, or additionally, a portion of stream 116 may be first sent to a second separator for removal of unreacted dehydrohalogenating agent, as described below, and then enter first distillation column 118.

Top stream 120 from first distillation column 118 may be removed and collected and/or sent to a subsequent reactor (not shown) for further processing. Bottom stream 122 from first distillation column 118 is sent to second distillation column 124. Second distillation column 124 operates as the product recovery column, separate HFO-1132 (Z) from unreacted HFC-143, as well as other recyclable and/or undesirable products. The top stream 126 from the second distillation column 124 contains a more concentrated or purified composition of HFO-1132 (Z) with a purity of HFO-1132 (Z) greater than 97 wt. %, greater than 98 wt. %, or greater than 99 wt. %, with trace amounts of impurities, as described in further detail herein. As also described further herein, the product stream 126 may be used as a reactant stream in the isomerization reaction of Step (iii). The bottom stream 128 from the second distillation column 124 contains HFC-143 with a purity greater than 99.3%. The bottom stream 128 enters third distillation column 130 to separate HFC-143 from the heavier side products and impurities. Top stream 136, which comprises a high purity of HFC-143, is recycled into reactor 114. The recycling of HFC-143 increases the efficiency of the overall reaction process. Bottom stream 134 from third distillation column 130 is disposed of, recovered, or recycled.

The dehydrochlorination reaction of Step (i) and dehydrofluorination reaction of Step (ii) may both be carried out in liquid phase in one or more of suitable reactors operated in series or parallel, for example reactors made from materials resistant to effects of temperature, pressure and/or corrosion including but not limited to stainless steels, duplex stainless steels, nickel and its alloys, including Hastelloy® (for example, Hastelloy C276), Inconel® (for example Inconel 600), Incoloy® (for example Incoloy 825), and Monel® or wherein the vessels which may be lined with fluoropolymers.

The liquid-phase reactor may be first cleaned and flushed with water, followed by charging with a basic solution made from metal hydroxides (M(OH)ₙ), for example, alkali metal hydroxides (e.g., LiOH, NaOH, KOH) or alkaline earth metal hydroxides (e.g., Mg(OH)₂, Ca(OH)₂, Sr(OH)₂), calcium oxide, or mixtures thereof.

Products may be flowed through one or more dryer columns to remove moisture. The reaction products may be collected by capture in a cooled cylinder, for example.

In some cases, Reactor 114 may generally be the same as the preconditioning reactor, but operated at a higher temperature (e.g., 5 °C higher, 10 °C higher). A two-reactor system may offer several advantages. For example, operating the preconditioning reactor at a lower temperature enables partial recovery of HFO-1123 and CTFE formed during Step (i), which would have been destroyed at higher temperatures required for defluorination reactions. In addition, since performing defluorination reaction in the presence of high concentrations of HCFCs will decrease the process yield due to formation of heavies, the preconditioning reactor is configured to decrease the concentration of HCFCs in the feed stream to increase overall process yield. However, a two-reactor system may not be required, such as when the reactant stream 112 comprises a substantially pure feed of HFC-143.

The process described herein may be conducted in a continuous reaction system, a batch reaction system, or a semi-continuous reaction system. The reaction may be carried out in a plurality of reactors in series or in parallel. The reaction conditions for each of Step (i) and Step (ii) in Process 1 are discussed in more details below.

### IV. Step (i)

As discussed above, the feed for Step (i) of Process 1 comprises HFC-143 and one or more intermediates HCFC-133b, HCFC-133, and/or HCFC-123a, and undesired byproducts including but not limited to HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

Since these intermediates and byproducts are difficult to separate from HFC-143, and their presence in the feed leads to a drop in selectivity to HFO-1132 (Z/E), the purpose of Step (i) is to convert these intermediates and byproducts to other components (e.g., CTFE, HFO-1123) which may be more easily separated from the product composition, thus producing a composition with higher purity of HFC-143. In Step (i) of Process 1, a feed including HFC-143 and one or more of the intermediates (e.g., HCFC-133b, HCFC-133, and/or HCFC-123a) reacts with a base where the one or more intermediates are dehydrochlorinated, then separated from the product stream to increase the purity of HFC-143 in the composition. The reaction condition of Step (i) may be selectively tailored to minimize conversion of HFC-143 while maximizing conversion of intermediates (e.g., HCFC-133b, HCFC-133, and/or HCFC-123a) and byproducts.

### Step (i) Reactant Composition

The reactant composition of Step (i) comprising HFC-143 may be obtained from a variety of ways, including but not limited to hydrogenating 1,1,2-trichloro-1,2,2-trifluoroethane (CFC-113), chlorotrifluoroethylene (CTFE) and/or trifluoroethylene (HFO-1123).

For example, the reactant composition of Step (i) may be obtained by reacting 1,1,2-trichloro-1,2,2-trifluoroethane (CFC-113) with hydrogen in the presence of a first catalyst at a temperature of from 150 °C to 400 °C to produce the first reactant composition comprising at least 60 mol % and less than or equal to 80 mol % HFC-143, based on total moles of organic components of the first reactant composition. For example, the reactant composition of Step (i) may be obtained by reacting CTFE (CFO-1113) with hydrogen in the presence of a second catalyst at a temperature of from 75 °C to 400 °C to produce the first reactant composition comprising at least 60 mol % and less than or equal to 95 mol % HFC-143, based on total moles of organic components of the first reactant composition. The first and catalyst used in the hydrogenation reaction to produce the reactant composition of Step (i) may be the same or different.

Suitable catalysts for use in the hydrogenation reaction to produce the reactant composition of Step (i) may include, for example, a metal such as palladium (Pd), platinum (Pt), rhodium (Rh), iridium (Ir), nickel (Ni), cobalt (Co), iron (Fe), ruthenium (Ru), or a combination thereof. The hydrogenation catalysts may be supported on common catalyst supports including, for example, activated carbon, porous aluminosilicate typified by zeolite, aluminum oxide (alumina), silicon oxide, titanium oxide, zirconium oxide, zinc oxide, aluminum fluoride, and the like.

The amount of HFC-143 in the reactant composition of Step (i) may be less than about 95 mol %, or less than about 93 mol %, or less than about 90 mol %, or less than about 85 mol %, or less than about 80 mol %, or less than about 75 mol %, or less than about 72 mol %, or less than about 70 mol %, or less than about 68 mol %, or less than about 65 mol %, or less than about 60 mol %, and for each of the foregoing, greater than 0.01 mol %, for example, based on total moles of organic components of the composition.

The combined amount of reactants other than HFC-143 in the reactant composition of Step (i) may be at least 5 mol %, at least 7 mol %, at least 10 mol %, at least 15 mol %, at least 20 mol %, at least 25 mol %, or at least 30 mol %, and for each of the foregoing, less than 99.99 mol %, for example, based on total moles of organic components of the composition.

For example when pure CFC-113 is used as the feed generating stream, the amount of HFC-143 in the reactant composition of Step (i) may be at least 60 mol % and less than or equal to 80 mol %, the amount of HCFC-133b may be at least 12 mol % and less than or equal to 20 mol %, the amount of HCFC-133 may be at least 1 mol % and less than or equal to 5 mol %, and the amount of HCFC-123a may be at least 7 mol % and less than or equal to 15 mol %, based on the combined total moles of the HFC-143, HCFC-133b, HCFC-133, and HCFC-123a in the reactant composition.

The combined amount of HFC-143, HCFC-133b, HCFC-133, and HCFC-123a in the reactant composition set forth in the preceding paragraph may be at least 85 mol % and less than or equal to 100% of the total moles of organic components of the product composition in the feed stream to the preconditioning reactor, while the combined amount of other components (e.g., HFC-152a) may be greater than or equal to 0 mol % and less than 15 mol % of the total moles of organic components of the product composition in the feed stream to the preconditioning reactor.

For example, when pure CTFE is used as the feed generating stream, the amount of HFC-143 in the reactant composition of Step (i) may be at least 60 mol % and less than or equal to 95 mol %, the amount of HCFC-133b may be at least 1 mol % and less than or equal to 5 mol %, the amount of HCFC-133 may be at least 1 mol % and less than or equal to 8 mol %, and the amount of HCFC-123a may be at least 0.01 mol % and less than or equal to 0.5 mol %, based on the combined total moles of the HFC-143, HCFC-133b, HCFC-133, and HCFC-123a in the reactant composition.

The combined amount of HFC-143, HCFC-133b, HCFC-133, and HCFC-123a in the reactant composition set forth in the preceding paragraph may be at least 85 mol % and less than or equal to 100% of the total moles of organic components of the product composition in the feed stream to the preconditioning reactor, while the combined amount of other components (e.g., HFC-152a) may be greater than or equal to 0 mol % and less than 15 mol % of the total moles of organic components of the product composition in the feed stream to the preconditioning reactor.

### Step (i) Reaction Base

In the liquid phase dehydrochlorination reaction of Step (i), HCFC-133b, HCFC-133, HCFC-123a, HFC-152a, and HCFC-142 isomers can be reacted with a suitable base to form CTFE, HFO-1123 and other byproducts. Suitable bases include, but are not limited to, at least one of potassium hydroxide (KOH), sodium hydroxide (NaOH), calcium oxide (CaO), calcium hydroxide (Ca(OH)₂), and/or combinations of the foregoing.

### Step (i) Reaction Temperature

The reaction temperature of the liquid phase dehydrochlorination reaction of Step (i) may be as low as about 80 °C, about 90 °C, about 95 °C, about 100 °C, about 102 °C or as high as about 104 °C, about 106 °C, about 108 °C, about 110 °C, about 120 °C, about 130 °C, or within any range encompassed by two of the foregoing values as endpoints, for example from about 80 °C to about 130 °C, or from about 90 °C to about 120 °C. The temperature may be preferably from about 95 °C to about 120 °C, and more preferably from about 95 °C to about 110 °C. Specific examples of additional suitable ranges are set forth below in Table 1. The numerical ranges set forth in Table 1 below are understood to be prefaced by “about”.

TABLE 1
Step (i) Reaction Temperature

| From (°C) | To (°C) |
|---|---|
| 80 | 104 |
| 80 | 106 |
| 80 | 108 |
| 80 | 110 |
| 80 | 120 |
| 80 | 130 |
| 90 | 104 |
| 90 | 106 |
| 90 | 108 |
| 90 | 110 |
| 90 | 120 |
| 90 | 130 |
| 95 | 104 |
| 95 | 106 |
| 95 | 108 |
| 95 | 110 |
| 95 | 120 |
| 95 | 130 |
| 100 | 104 |
| 100 | 106 |
| 100 | 108 |
| 100 | 110 |
| 100 | 120 |
| 100 | 130 |
| 102 | 104 |
| 102 | 106 |
| 102 | 108 |
| 102 | 110 |
| 102 | 120 |
| 102 | 130 |

### Step (i) Base to Reactant Mole Ratio

The mole ratio of base (OH⁻) to reactant (e.g., HFC-143, HCFC-133b, HCFC-133, and HCFC-123a) of the liquid phase dehydrochlorination reaction of Step (i) may be as low as about 0.1, about 0.2, about 0.3, about 0.4, about 0.5, about 0.6, about 0.7, or as high as about 0.8, about 0.9, about 1.0, about 1.1, about 1.2, about 1.3, about 1.4, about 1.5, or within any range encompassed by two of the foregoing values as endpoints, for example from about 0.1 to about 1.5, or from about 0.2 to about 1.4. The mole ratio of base to reactant (HFC-143, HCFC-133b, HCFC-133, and HCFC-123a) may preferably be from about 0.2 to about 1.3, and more preferably from about 0.2 to about 1.2.

The mole ratio of base (OH⁻) to reactant (e.g., HCFC-133b, HCFC-133, and HCFC-123a) of the liquid phase dehydrochlorination reaction of Step (i) may be as low as about 0.5, about 1.0, about 1.5, about 2.0, about 2.5, or as high as about 3.0, about 3.5, about 4.0, about 4.5, about 5.0, about 5.5, about 6.0, about 6.5, about 7.0, about 8.0, about 9.0, about 10.0, or within any range encompassed by two of the foregoing values as endpoints, for example from about 0.5 to about 10.0, or from about 1.0 to about 9.0. The mole ratio of base to reactant (HCFC-133b, HCFC-133, and HCFC-123a) may preferably be from about 1.0 to about 8.0, and more preferably from about 1.0 to about 6.0. Specific examples of additional suitable ranges are set forth below in Table 2. The numerical ranges set forth in Table 2 below are understood to be prefaced by “about”.

The base containing feed stream can be an aqueous mixture and can contain the base at any suitable strength for producing the desired reaction. For example, when KOH is used as the base, the base containing feed stream can contain base at a strength from about 5 wt. % to about 50 wt. %, or from about 10 wt. % to about 45 wt. %, or preferably from about 20 wt. % to about 45 wt. %. Optionally, a phase transfer agent or catalyst, for example, alcohol and Aliquat®336 (C25H54ClN) can be added to the reaction in the at least one reactor, which can enhance the reaction rate. Other phase transfer catalysts that may be used include, but are not limited to, quaternary ammonium salts, quaternary phosphonium salts, quaternary arsenium salts, sulfonium salts, crown ethers and similar molecules. A quaternary ammonium salt or a quaternary phosphonium salt is more preferred.

TABLE 2
Step (i) Base (OH⁻) to Reactant (e.g.,
HCFC-133b, HCFC-133, and HCFC-123a) Mole ratio

| From | To |
|---|---|
| 0.5 | 3.0 |
| 0.5 | 4.0 |
| 0.5 | 5.0 |
| 0.5 | 6.0 |
| 0.5 | 7.0 |
| 0.5 | 8.0 |
| 0.5 | 9.0 |
| 0.5 | 10.0 |
| 1.0 | 3.0 |
| 1.0 | 4.0 |
| 1.0 | 5.0 |
| 1.0 | 6.0 |
| 1.0 | 7.0 |
| 1.0 | 8.0 |
| 1.0 | 9.0 |
| 1.0 | 10.0 |
| 1.5 | 3.0 |
| 1.5 | 4.0 |
| 1.5 | 5.0 |
| 1.5 | 6.0 |
| 1.5 | 7.0 |
| 1.5 | 8.0 |
| 1.5 | 9.0 |
| 1.5 | 10.0 |
| 2.0 | 3.0 |
| 2.0 | 4.0 |
| 2.0 | 5.0 |
| 2.0 | 6.0 |
| 2.0 | 7.0 |
| 2.0 | 8.0 |
| 2.0 | 9.0 |
| 2.0 | 10.0 |

### Step (i) Base Weight Percentage

The weight percentage of the base used for the liquid phase dehydrochlorination reaction of Step (i) may be as low as about 5 wt. %, about 10 wt. %, about 15 wt. %, about 20 wt. %, about 25 wt. %, or as high as about 30 wt. %, about 35 wt. %, about 40 wt. %, about 45 wt. %, about 50 wt. %, about 55 wt. %, about 60 wt. %, or within any range encompassed by two of the foregoing values as endpoints, for example from about 5 wt. % to about 50 wt. %, based on total weight of the base. The weight percentage of the base may preferably be from about 10 wt. % to about 45 wt. %, based on total weight of the base. Specific examples of additional suitable ranges are set forth below in Table 3. The numerical ranges set forth in Table 3 below are understood to be prefaced by “about”.

TABLE 3
Step (i) Base Weight Percentage

| From (%) | To (%) |
|---|---|
| 5 | 30 |
| 5 | 35 |
| 5 | 40 |
| 5 | 45 |
| 5 | 50 |
| 5 | 55 |
| 5 | 60 |
| 10 | 30 |
| 10 | 35 |
| 10 | 40 |
| 10 | 45 |
| 10 | 50 |
| 10 | 55 |
| 10 | 60 |
| 15 | 30 |
| 15 | 35 |
| 15 | 40 |
| 5 | 45 |
| 5 | 50 |
| 15 | 55 |
| 15 | 60 |
| 20 | 30 |
| 20 | 35 |
| 20 | 40 |
| 20 | 45 |
| 20 | 50 |
| 20 | 55 |
| 20 | 60 |
| 25 | 30 |
| 25 | 35 |
| 25 | 40 |
| 25 | 45 |
| 25 | 50 |
| 25 | 55 |
| 25 | 60 |

### Step (i) Reaction Conditions

A summary of the preferred base, temperatures, base weight percentages, and base (OH⁻) to reactant (e.g., HCFC-133b, HCFC-133, and HCFC-123a) mole ratio as discussed above are summarized in Table 4 below. The numerical ranges set forth in Table 4 below are understood to be prefaced by “about”.

TABLE 4
Summary of Preferred Step (i) Reaction Conditions

| | Base Weight | | |
|---|---|---|---|
| | Percentage | Mole | Temperature |
| Base | (wt. %) | ratio* | (°C) |
| KOH | 5-60 | 0.5-10 | 80-130 |
| KOH | 5-60 | 0.5-10 | 85-120 |
| KOH | 5-60 | 0.5-10 | 90-115 |
| KOH | 5-60 | 0.5-10 | 95-110 |
| KOH | 5-60 | 1-8 | 80-130 |
| KOH | 5-60 | 1-8 | 85-120 |
| KOH | 5-60 | 1-8 | 90-115 |
| KOH | 5-60 | 1-8 | 95-110 |
| KOH | 5-60 | 1-6 | 80-130 |
| KOH | 5-60 | 1-6 | 85-120 |
| KOH | 5-60 | 1-6 | 90-115 |
| KOH | 5-60 | 1-6 | 95-110 |
| KOH | 10-55 | 0.5-10 | 80-130 |
| KOH | 10-55 | 0.5-10 | 85-120 |
| KOH | 10-55 | 0.5-10 | 90-115 |
| KOH | 10-55 | 0.5-10 | 95-110 |
| KOH | 10-55 | 1-8 | 80-130 |
| KOH | 10-55 | 1-8 | 85-120 |
| KOH | 10-55 | 1-8 | 90-115 |
| KOH | 10-55 | 1-8 | 95-110 |
| KOH | 10-55 | 1-6 | 80-130 |
| KOH | 10-55 | 1-6 | 85-120 |
| KOH | 10-55 | 1-6 | 90-115 |
| KOH | 10-55 | 1-6 | 95-110 |

*Mole ratio denotes the mole ratio of base (OH⁻) to reactant (e.g., HCFC-133b, HCFC-133, and HCFC-123a)

### Step (i) Conversion

As demonstrated by the Examples herein, for the liquid phase dehydrochlorination reaction of Step (i), the reaction may achieve a conversion of HCFC-123a of at least about 80%, at least about 85%, at least about 90%, or at least about 95%, and for each of the foregoing, less than or equal to 100%, or within any range encompassed by two of the foregoing values as endpoints, for example from about 80% to about 100%, or from about 90% to about 100%, based on total moles of the organic components of the composition.

As demonstrated by the Examples herein, for the liquid phase dehydrochlorination reaction of Step (i), the reaction may achieve a conversion of HCFC-133 and HCFC-133b of at least about 20%, at least about 60%, at least about 80%, or at least about 90%, and for each of the foregoing, less than or equal to 100%, or within any range encompassed by two of the foregoing values as endpoints, for example from about 20% to about 100%, or from about 60% to about 100%, based on total moles of the organic components of the composition.

As demonstrated by the Examples herein, for the liquid phase dehydrochlorination reaction of Step (i), the reaction may achieve a conversion of HFC-143 of less than about 25%, less than about 20%, less than about 15%, less than about 10%, or less than about 5%, and for each of the foregoing, greater than or equal to 0%, or within any range encompassed by two of the foregoing values as endpoints, for example from about 0% to about 25%, or from about 0% to about 20%, based on total moles of the organic components of the composition.

### Step (i) Selectivity

As demonstrated by the Examples herein, for the liquid phase dehydrochlorination reaction of Step (i), the reaction may achieve a selectivity towards CTFE of at least about 5%, at least about 15%, at least about 25%, at least about 35%, at least about 45%, at least about 55%, at least about 65%, at least about 75%, at least about 85%, at least about 90%, and for each of the foregoing, less than or equal to 100%, or within any range encompassed by two of the foregoing values as endpoints, for example from about 5% to about 100%, or from about 15% to about 100%, based on total moles of the organic components of the composition.

As demonstrated by the Examples herein, for the liquid phase dehydrochlorination reaction of Step (i), the reaction may achieve a selectivity towards HFC-1123 of at least about 10%, at least about 20%, at least about 30%, at least about 40%, at least about 50%, at least about 60%, at least about 70%, at least about 80%, at least about 90%, at least about 95%, and for each of the foregoing, less than or equal to 100%, or within any range encompassed by two of the foregoing values as endpoints, for example from about 10% to about 100%, or from about 20% to about 100%, based on total moles of the organic components of the composition.

As demonstrated by the Examples herein, for the liquid phase dehydrochlorination reaction of Step (i), the reaction may achieve a selectivity towards HFO-1132 of less than 20%, less than 15%, less than 10%, less than 5%, and for each of the foregoing, greater than or equal to 0%, or within any range encompassed by two of the foregoing values as endpoints, for example from about 0% to about 20%, or from about 0% to about 15%, based on total moles of the organic components of the composition.

### Step (i) Product Stream Composition

For the liquid phase dehydrochlorination reaction of Step (i), the amount of HFC-143 in the first product stream from the preconditioning reactor may be at least 60 mol %, at least 70 mol %, at least 75 mol %, at least 80 mol %, and for each of the foregoing, less than or equal to 100 mol %, for example, based on total moles of organic components of the composition.

For the liquid phase dehydrochlorination reaction of Step (i), the amount of HCFC-133b in the first product stream from the preconditioning reactor, if present, may be less than 15 mol %, less than 10 mol %, less than 5 mol %, less than 3 mol %, and for each of the foregoing, great than or equal to 0 mol %, for example, based on total moles of organic components of the composition.

For the liquid phase dehydrochlorination reaction of Step (i), the amount of HCFC-133 in the first product stream from the preconditioning reactor, if present, may be less than 8 mol %, less than 7 mol %, less than 6 mol %, less than 5 mol %, less than 4 mol %, less than 3 mol %, less than 2 mol %, less than 1 mol %, and for each of the foregoing, great than or equal to 0 mol %, for example, based on total moles of organic components of the composition.

For the liquid phase dehydrochlorination reaction of Step (i), the amount of HCFC-123a in the first product stream from the preconditioning reactor, if present, may be less than 10 mol %, less than 9 mol %, less than 8 mol %, less than 7 mol %, less than 6 mol %, less than 5 mol %, less than 4 mol %, less than 3 mol %, less than 2 mol %, less than 1 mol %, less than 0.5 mol %, less than 0.3 mol %, and for each of the foregoing, great than or equal to 0 mol %, for example, based on total moles of organic components of the composition.

For example, for a liquid phase dehydrochlorination reaction of Step (i), in the first product stream from the preconditioning reactor, the amount of HFC-143 may be at least 70 mol % and less than or equal to 99.8 mol %, the amount of HCFC-133b may be at least 0.1 mol % and less than or equal to 15 mol %, the amount of HCFC-133 may be at least 0.1 mol % and less than or equal to 7 mol %, and the amount of 1,2-dichloro-1,1,2-trifluoroethane (HCFC-123a) may be at least 0 mol % and less than or equal to 8 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 80 mol % and less than or equal to 100% of the total moles of organic components of the product composition in the product stream from the preconditioning reactor, while the combined amount of other components including undesired byproducts (e.g., HCC-160, HFC-152a, and HCFC-142 isomers) may be greater than or equal to 0 mol % and less than 20 mol % of the total moles of organic components of the product composition in the product stream from the preconditioning reactor.

The product stream may also include a salt by-product of the dehydrohalogenating agent from the dehydrohalogenation reaction. The salt by-product may be isolated from the product stream using known techniques and converted back to the dehydrohalogenating agent using known methods. It may then be recycled to the dehydrohalogenation reaction independently for the step (i) reaction. Specifically, the salt by-product and optionally the unreacted dehydrohalogenating agent in the product stream may be separated from organics in the product stream via a separator before the salt by-product is converted back to the dehydrohalogenating agent and then returned to the preconditioning reactor via a return line, wherein the foregoing occurs before the product stream enters the preconditioning distillation column. Alternatively, the dehydrohalogenating agent (resulting from the conversion of the salt by-product and the unreacted dehydrohalogenating agent) in the product stream may be separated from organics in the product stream after the salt by-product is converted back to the dehydrohalogenating agent and before the product stream enters the preconditioning distillation column.

### Step (i) Post Processing

As discussed above, the product stream from the preconditioning reactor enters the preconditioning distillation column to remove lights from the first product composition. The top stream from the preconditioning distillation column containing CTFE, HFO-1132 isomers, and HFO-1123 may be recycled or sent to a reactor producing the feed stream. Suitable column conditions for the preconditioning distillation column are as follows.

The number of stages for the preconditioning distillation column may be as low as about 20, about 22, about 24, about 26, about 28, or as high as about 32, about 34, about 36, about 38, about 40, or within any range encompassed by any two of the foregoing values as endpoints, for example, from about 20 to about 40, or from about 22 to about 38. The number of stages for the preconditioning distillation column may be preferably from about 24 to about 36, or more preferably from about 26 to about 34.

The temperature within the preconditioning distillation column may be as low as about −10 °C, about −0 °C, about 10 °C, about 20 °C, about 30 °C, about 40 °C, about 50 °C, about 60 °C, about 70 °C, about 80 °C, about 90 °C, about 100 °C, about 120 °C or as high as about 130 °C, about 140 °C, about 150 °C, about 160 °C, about 170 °C, about 180 °C, about 190 °C, about 200 °C or within any range encompassed by two of the foregoing values as endpoints. The temperature may be preferably from about 0 °C to about 50 °C, and more preferably from about 10 °C to about 30 °C.

The pressure within the preconditioning distillation column may be as low as about 10 torr, about 50 torr, about 100 torr, about 300 torr, about 500 torr, about 700 torr or as high as about 0 psig, about 15 psig, about 30 psig, about 50 psig, about 70 psig, about 100 psig, about 150 psig, about 200 psig or any range using any of the foregoing values as endpoints. The pressure may preferably be from about 700 torr to about 50 psig, and more preferably be from about 0 psig to about 50 psig.

For the liquid phase dehydrochlorination reaction of Step (i), the amount or purity of HFC-143 in the bottom stream from the preconditioning distillation column may be at least 90 mol %, at least 92 mol %, at least 94 mol %, or at least 95 mol %, and for each of the foregoing, less than or equal to 100 mol %, for example, based on total moles of organic components in the composition.

For the liquid phase dehydrochlorination reaction of Step (i), the combined amount of HCFC-133b, HCFC-133, and HCFC-123 in the stream 112 preconditioning distillation column, if present, may be less than 10 mol %, less than 8 mol %, less than 6 mol %, less than 5 mol %, and for each of the foregoing, great than or equal to 0 mol %, for example, based on total moles of organic components of the composition.

For the liquid phase dehydrochlorination reaction of Step (i), the amount of HFC-143 in the top stream from the preconditioning distillation column, if present, may be less than 15 mol %, less than 14 mol %, less than 13 mol %, less than 12 mol %, and for each of the foregoing, greater than or equal to 0 mol %, for example, based on total moles of organic components of the composition.

For the liquid phase dehydrochlorination reaction of Step (i), the combined amount of CTFE and HFO-1123 in the top stream from the preconditioning distillation column may be at least 50 mol %, at least 55 mol %, at least 60 mol %, or at least 65 mol %, and for each of the foregoing, less than or equal to 100 mol %, for example, based on total moles of organic components in the composition.

Exemplary compositions for the stream 112 preconditioning distillation columnare set forth below in Table 5. The numerical ranges set forth in Table 5 below are understood to be prefaced by “about”.

TABLE 5
Composition of Stream 112

| | | | | | | | Combined |
|---|---|---|---|---|---|---|---|
| | | | | | | | amount of R-143, |
| | | | | | | | R-133b, R-133, |
| | | | | | | | and R-123 in the |
| Comp. | R-143 | R-133b | | R-133 | | R-123a | composition |
| 1 | 94-99.8 wt % | 0.1-5 | wt % | 0.1-0.5 | wt % | 0-0.5 wt % | 95-100 wt % |
| 2 | 95-99.8 wt % | 0.1-4 | wt % | 0.1-0.5 | wt % | 0-0.5 wt % | 95-100 wt % |
| 3 | 99-99.9 wt % | 0.1-0.8 | wt % | 0-0.1 | wt % | 0-0.1 wt % | 99-100 wt % |

### V. Step (ii)

Referring again to FIG. 1, reactant stream 112 contains a high purity of HFC-143, and enters a preheater 115 to heat the stream 112 before entering second reactor 114. Stream 112 is combined with a base feed stream 113 and the combined stream 111 enters reactor 114. Inside reactor 114, HFC-143 undergoes dehydrofluorination reaction of Step (ii) in liquid phase to produce a second product composition comprising HFO-1132 (Z/E).

### Step (ii) Reaction Feed

As described previously, in certain embodiments, the reactant composition provided to Step (ii) comprises liquid 1,1,2-trifluoroethane (HFC-143) having a purity of at least 95.0 wt. %, 99.0 wt. %, at least 99.5 wt. %, or at least 99.9 wt. %, as based on total weight of organic components. In other embodiments, the reactant composition provided to Step (ii) comprises liquid 1,1,2-trifluoroethane (HFC-143) and one or more additional organic components, including one or more intermediates, undesired byproducts, or reaction byproducts, such as HCFC-133b, HCFC-133, HCFC-123a, HFC-152a, HFO-1123, HFO-1132a, HFO-1132 (Z), HFO-1132(E), or combinations thereof. Here, the reactant composition may be introduced batchwise or semi-batchwise into a reaction vessel optionally equipped with any one of or combination of agitation, a heating and cooling jacket, and an integrated reactor distillation column and condenser, thereby permitting reaction and simultaneous separation of one or more volatile components during Step (ii).

### Step (ii) Reaction Base

In the liquid phase dehydrofluorination reaction of Step (ii), HFC-143 can be reacted with a suitable base to form HFO-1132 (Z), HFO-1132(Z), HFO-1132a, and other byproducts. Suitable bases include, but are not limited to, potassium hydroxide (KOH), sodium hydroxide (NaOH), calcium oxide (CaO), and/or calcium hydroxide (Ca(OH)₂). Additionally, the base may be used in combination with one or more diluents, solvents and/or reactants, such as one or more alcohols including, for example, methanol, ethanol, propanol, butanol, isopropyl alcohol, and/or tert-butyl alcohol.

### Step (ii) Reaction Temperature

The reactants may reach a target temperature in the preheater 115 or in the reactor 114. In one example, the preheater 115 brings reactants up to a first temperature and the reactor 114 maintains the reaction at the first temperature. In another example, the reactor 114 brings the reactants up to a second temperature and maintains the reaction at the second temperature, wherein the second temperature is greater than or less than the first temperature. The reaction temperature of the liquid phase dehydrofluorination reaction of Step (ii) may be as low as about 90 °C, about 95 °C, about 100 °C, about 105 °C, about 110 °C, about 115 °C, or as high as about 120 °C, about 125 °C, about 130 °C, about 135 °C, about 140 °C, about 150 °C, or within any range encompassed by two of the foregoing values as endpoints, for example from about 90 °C to about 150 °C, or from about 95 °C to about 140 °C. Specific examples of additional suitable ranges are set forth below in Table 6. The numerical ranges set forth in Table 6 below are understood to be prefaced by “about”.

TABLE 6
Step (ii) Reaction Temperature

| From (°C) | To (°C) |
|---|---|
| 90 | 120 |
| 90 | 125 |
| 90 | 130 |
| 90 | 135 |
| 90 | 140 |
| 90 | 150 |
| 95 | 120 |
| 95 | 125 |
| 95 | 130 |
| 95 | 135 |
| 95 | 140 |
| 95 | 150 |
| 100 | 120 |
| 100 | 125 |
| 100 | 130 |
| 100 | 135 |
| 100 | 140 |
| 100 | 150 |
| 105 | 120 |
| 105 | 125 |
| 105 | 130 |
| 105 | 135 |
| 105 | 140 |
| 105 | 150 |
| 110 | 120 |
| 110 | 125 |
| 110 | 130 |
| 110 | 135 |
| 110 | 140 |
| 110 | 150 |
| 115 | 120 |
| 115 | 125 |
| 115 | 130 |
| 115 | 135 |
| 115 | 140 |
| 115 | 150 |

### Step (ii) Base to Reactant Mole Ratio

The mole ratio of base to reactant (HFC-143, HCFC-133b, HCFC-133, and HCFC-123a) of the liquid phase dehydrofluorination reaction of Step (ii) may be as low as about 0.1, about 0.2, about 0.3, about 0.4, about 0.5, about 0.6, about 0.7, about 0.8, about 0.9, about 1.0, or as high as about 1.1, about 1.2, about 1.3, about 1.4, about 1.5, about 1.6, about 1.7, about 1.8, about 1.9, about 2.0, about 3.0, about 4.0, about 5.0, or within any range encompassed by two of the foregoing values as endpoints, for example from about 0.1 to about 2.0, or from about 0.2 to about 1.6, preferably from about 1 to about 2. The mole ratio of base to reactant (HFC-143, HCFC-133b, HCFC-133, and HCFC-123a) may preferably be from about 0.8 to about 1.8, more preferably from about 1.0 to about 1.5, and most preferably from about 1.0 to about 1.2. Specific examples of additional suitable ranges are set forth below in Table 8. The numerical ranges set forth in Table 8 below are understood to be prefaced by “about”.

The base containing feed stream can be an aqueous mixture and can contain the base at any suitable strength for producing the desired reaction. For example, when KOH is used as the base, the base containing feed stream can contain base at a strength from about 5 wt. % to about 50 wt. %, preferably from about 20 wt. % to about 45 wt. %. Optionally, a phase transfer agent or catalyst, for example, Aliquat®336 (C₂₅H₅₄ClN) can be added to the reaction in the at least one reactor, which can enhance the reaction rate. Other phase transfer catalysts that can be used include, but not limited to, quaternary ammonium salts, quaternary phosphonium salts, quaternary arsenium salts, sulfonium salts, crown ethers and similar molecules. A quaternary ammonium salt or a quaternary phosphonium salt is more preferred. If a phase transfer catalyst is used, the base containing feed stream may comprise phase transfer catalyst in an amount greater than about 0.01 wt. %, about 0.02 wt. %, about 0.03 wt. %, about 0.04 wt. %, about 0.05 wt. %, or less than about 0.06 wt. %, about 0.07 wt. %, about 0.08 wt. %, about 0.09 wt. %, about 0.1 wt. %, about 1 wt. %, or within any range encompassed by two of the foregoing values as endpoints, for example from about 0.01 wt. % to about 1 wt. %, preferably from about 0.01 wt. % to about 0.1 wt. %, more preferably from about 0.03 wt. % to about 0.08 wt. %.

TABLE 7
Step (ii) Base (OH⁻) to Reactant (e.g., HFC-
143, HCFC-133b, HCFC-133, and HCFC-123a) Mole ratio

| From | To |
|---|---|
| 0.1 | 1.1 |
| 0.1 | 1.2 |
| 0.1 | 1.3 |
| 0.1 | 1.4 |
| 0.1 | 1.5 |
| 0.1 | 2.0 |
| 0.1 | 3.0 |
| 0.1 | 4.0 |
| 0.1 | 5.0 |
| 0.5 | 1.1 |
| 0.5 | 1.2 |
| 0.5 | 1.3 |
| 0.5 | 1.4 |
| 0.5 | 1.5 |
| 0.5 | 2.0 |
| 0.5 | 3.0 |
| 0.5 | 4.0 |
| 0.5 | 5.0 |
| 1.0 | 1.1 |
| 1.0 | 1.2 |
| 1.0 | 1.3 |
| 1.0 | 1.4 |
| 1.0 | 1.5 |
| 1.0 | 2.0 |
| 1.0 | 3.0 |
| 1.0 | 4.0 |
| 1.0 | 5.0 |

### Step (ii) Base Weight Percentage

The weight percentage of the base used for the liquid phase dehydrochlorination reaction of Step (i) may be as low as about 5 wt. %, about 10 wt. %, about 15 wt. %, about 20 wt. %, about 25 wt. %, or as high as about 30 wt. %, about 35 wt. %, about 40 wt. %, about 45 wt. %, about 50 wt. %, about 55 wt. %, about 60 wt. %, or within any range encompassed by two of the foregoing values as endpoints, for example from about 5 wt. % to about 50 wt. %, based on total weight of the base. The weight percentage of the base may preferably be from about 10 wt. % to about 60 wt. %, and more preferably from about 10 wt. % to about 45 wt. %, based on total weight of the base. Specific examples of additional suitable ranges are set forth below in Table 8. The numerical ranges set forth in Table 8 below are understood to be prefaced by “about”.

TABLE 8
Step (ii) Base Weight Percentage

| From (%) | To (%) |
|---|---|
| 5 | 30 |
| 5 | 35 |
| 5 | 40 |
| 5 | 45 |
| 5 | 50 |
| 5 | 55 |
| 5 | 60 |
| 10 | 30 |
| 10 | 35 |
| 10 | 40 |
| 10 | 45 |
| 10 | 50 |
| 10 | 55 |
| 10 | 60 |
| 15 | 30 |
| 15 | 35 |
| 15 | 40 |
| 5 | 45 |
| 5 | 50 |
| 15 | 55 |
| 15 | 60 |
| 20 | 30 |
| 20 | 35 |
| 20 | 40 |
| 20 | 45 |
| 20 | 50 |
| 20 | 55 |
| 20 | 60 |
| 25 | 30 |
| 25 | 35 |
| 25 | 40 |
| 25 | 45 |
| 25 | 50 |
| 25 | 55 |
| 25 | 60 |

### Step (ii) Base and Alcohol Combination

The base may be combined with an alcohol as a diluent, solvent, and/or reactant during step (ii). Suitable alcohols include C1-C4 alcohols, such as methanol, ethanol, propanol, butanol, isopropyl alcohol, tert-butyl alcohol, and combinations thereof. The alcohol may be used in place of, or in combination with, other solvents such as water.

The alcohol may be present in an amount sufficient to form a liquid reaction medium. In some cases, the alcohol is present in an amount of as little as about 10 wt. %, about 25 wt. %, about 50 wt. %, or about 75 wt. %, or as much as about 100 wt. %, about 150 wt. %, or about 200 wt. %, based on the weight of the reactant introduced into step (ii), or within any range using any two of the foregoing values as endpoints, such as from about 10 wt. % to about 200 wt. %.

TABLE 9
Alcohol wt. %

| From (%) | To (%) |
|---|---|
| 10 | 100 |
| 10 | 150 |
| 10 | 200 |
| 25 | 100 |
| 25 | 150 |
| 25 | 200 |
| 50 | 100 |
| 50 | 150 |
| 50 | 200 |
| 75 | 100 |
| 75 | 150 |
| 75 | 200 |

In terms of alcohol-to-reactant weight ratio, the alcohol-to-reactant weight ratio may be as little as about 0.1:1, about 0.3:1, about 0.5:1, or about 0.8:1, or as much as about 1.0:1, about 1.5:1, or about 2:1, or within any range using any two of the foregoing values as endpoints, such as from about 0.1:1 to about 2:1.

TABLE 10
Alcohol-to-Reactant Weight Ratio

| From (%) | To (%) |
|---|---|
| 0.1:1 | 1.0:1 |
| 0.1:1 | 1.5:1 |
| 0.1:1 | 2.0:1 |
| 0.3:1 | 1.0:1 |
| 0.3:1 | 1.5:1 |
| 0.3:1 | 2.0:1 |
| 0.5:1 | 1.0:1 |
| 0.5:1 | 1.5:1 |
| 0.5:1 | 2.0:1 |
| 0.8:1 | 1.0:1 |
| 0.8:1 | 1.5:1 |
| 0.8:1 | 2.0:1 |

The base may be provided as a solid or as a solution or slurry in the alcohol. Suitable bases include alkali metal hydroxides and alkoxides. The molar ratio of base to reactant may be as little as about 0.8:1, about 1.0:1, about 1.1:1, or about 1.2:1, or as much as about 1.4:1, about 2.0:1, or about 3.0:1, or within any range using any two of the foregoing values as endpoints, such as from about 0.8:1 to about 3.0:1

TABLE 11
Base-to-Reactant Molar Ratio

| From (%) | To (%) |
|---|---|
| 0.8:1 | 1.4:1 |
| 0.8:1 | 2.0:1 |
| 0.8:1 | 3.0:1 |
| 1.0:1 | 1.4:1 |
| 1.0:1 | 2.0:1 |
| 1.0:1 | 3.0:1 |
| 1.1:1 | 1.4:1 |
| 1.1:1 | 2.0:1 |
| 1.1:1 | 3.0:1 |
| 1.2:1 | 1.4:1 |
| 1.2:1 | 2.0:1 |
| 1.2:1 | 3.0:1 |

When step (ii) is conducted in the presence of an alcohol, the dehydrofluorination reaction may proceed with a conversion of 1,1,2-trifluoroethane (HFC-143) of as little as about 25%, about 35%, about 45%, or about 55%, or as much as about 60%, about 80%, or about 100%, or within any range using any two of the foregoing values as endpoints, such as from about 25% to about 100%, based on the total moles of HFC-143 introduced into the reaction.

TABLE 12
HFC-143 Conversion (%)

| From (%) | To (%) |
|---|---|
| 25 | 60 |
| 25 | 80 |
| 25 | 100 |
| 35 | 60 |
| 35 | 80 |
| 35 | 100 |
| 45 | 60 |
| 45 | 80 |
| 45 | 100 |
| 55 | 60 |
| 55 | 80 |
| 55 | 100 |

In alcohol-containing systems, the reaction may exhibit a selectivity toward 1,2-difluoroethylene isomers (HFO-1132(Z) and/or HFO-1132(E)) of as little as about 35%, about 50%, about 65%, or about 75%, or as much as about 80%, about 90%, or about 100%, or within any range using any two of the foregoing values as endpoints, such as from about 35% to about 100%, based on moles of converted HFC-143.

TABLE 13
Selectivity to HFO-1132 (%)

| From (%) | To (%) |
|---|---|
| 35 | 80 |
| 35 | 90 |
| 35 | 100 |
| 50 | 80 |
| 50 | 90 |
| 50 | 100 |
| 65 | 80 |
| 65 | 90 |
| 65 | 100 |
| 75 | 80 |
| 75 | 90 |
| 75 | 100 |

Minor amounts of additional reaction products may also be formed during step (ii), including vinylidene fluoride (HFO-1132a) and alcohol-derived species such as ethers or alkoxylated fluorocarbons. The selectivity to such minor products may be as little as about 0.1%, about 0.5%, or about 1%, or as much as about 2%, about 5%, or about 10%, or within any range using any two of the foregoing values as endpoints, such as from about 0.1% to about 10%, based on converted HFC-143.

TABLE 14
Selectivity to Minor Products (%)

| From (%) | To (%) |
|---|---|
| 0.1 | 2 |
| 0.1 | 5 |
| 0.1 | 10 |
| 0.5 | 2 |
| 0.5 | 5 |
| 0.5 | 10 |
| 1.0 | 2 |
| 1.0 | 5 |
| 1.0 | 10 |
| 0.1 | 2 |
| 0.1 | 5 |
| 0.1 | 10 |

### Step (ii) Reaction Conditions

A summary of the preferred base, temperatures, base weight percentages, and base (OH) to reactant (e.g., HFC-143, HCFC-133b, HCFC-133, and HCFC-123a) mole ratio as discussed above are summarized in Table 10 below. The numerical ranges set forth in Table 10 below are understood to be prefaced by “about”.

TABLE 15
Summary of Preferred Step (ii) Reaction Conditions

| | Base Weight Percentage | | |
|---|---|---|---|
| Base | (wt. %) | Mole ratio* | Temperature (°C) |
| KOH | 5-60 | 0.1-4 | 90-150 |
| KOH | 5-60 | 0.1-4 | 95-140 |
| KOH | 5-60 | 0.1-4 | 100-130 |
| KOH | 5-60 | 0.1-4 | 100-120 |
| KOH | 5-60 | 0.5-3 | 90-150 |
| KOH | 5-60 | 0.5-3 | 95-140 |
| KOH | 5-60 | 0.5-3 | 100-130 |
| KOH | 5-60 | 0.5-3 | 100-120 |
| KOH | 5-60 | 1-2 | 90-150 |
| KOH | 5-60 | 1-2 | 95-140 |
| KOH | 5-60 | 1-2 | 100-130 |
| KOH | 5-60 | 1-2 | 100-120 |
| KOH | 10-55 | 0.1-4 | 90-150 |
| KOH | 10-55 | 0.1-4 | 95-140 |
| KOH | 10-55 | 0.1-4 | 100-130 |
| KOH | 10-55 | 0.1-4 | 100-120 |
| KOH | 10-55 | 0.5-3 | 90-150 |
| KOH | 10-55 | 0.5-3 | 95-140 |
| KOH | 10-55 | 0.5-3 | 100-130 |
| KOH | 10-55 | 0.5-3 | 100-120 |
| KOH | 10-55 | 1-2 | 90-150 |
| KOH | 10-55 | 1-2 | 95-140 |
| KOH | 10-55 | 1-2 | 100-130 |
| KOH | 10-55 | 1-2 | 100-120 |

*Mole ratio denotes the mole ratio of base (OH⁻) to reactant (e.g., HFC-143, HCFC-133b, HCFC-133, and HCFC-123a)

### Step (ii) Conversion of HFC-143

As demonstrated by the Examples herein, for the liquid phase dehydrofluorination reaction of Step (ii), the reaction may achieve a conversion of HFC-143 of at least 25%, at least about 30%, at least about 35%, or at least about 40%, and for each of the foregoing, less than or equal to 100%, or within any range encompassed by two of the foregoing values as endpoints, for example from about 25% to about 100%, or from about 35% to about 100%, based on total moles of the organic components of the composition. The reaction may be considered complete by an increase in condenser temperature to approximately 60-80 °C, reflecting depletion of volatile components in the reactor overhead.

Step (ii) Selectivity As demonstrated by the Examples herein, for the liquid phase dehydrofluorination reaction of Step (ii), the reaction may achieve a selectivity towards HFO-1132 isomers of at least 35%, at least about 45%, at least about 55%, or at least about 65%, and for each of the foregoing, less than or equal to 100%, or within any range encompassed by two of the foregoing values as endpoints, for example from about 35% to about 100%, or from about 65% to about 100%, based on total moles of the organic components of the composition.

### Step (ii) Side Products

At least one side reaction may occur during the dehydrofluorination reaction of Step (ii), which may create various side products alongside the formation of HFO-1132 isomers. The side products may include at least one of HFO-1132a, R-1123, R-1113, R-1112, CFCl═CHF (R-1122a), CF₂═CHCl (R-1122), CHCl═CHF (R-1131), CH₂═CHF (R-1141), CF≡CH, CCl≡CH, difluoroethanol, 2,2-difluoroethanol, dimers/trimers/oligomers, and KF. Examples of potential side product reactions from HFC-143 and reaction products are shown below, where M is K, Na, or Ca, and n is 1 or 2:
(ii)-a CHF2CH2F (R-143)+M(OH)ₙ→CF2=CH₂ (HFO-1132a)+MFₙ+H2O
(ii)-b CHF2CH2F (R-143)+M(OH)ₙ→CHF2CH2OH (2,2-difluoroethanol)+MFₙ
   (ii)-c HFO-1132E/Z/HFO-1132a+M(OH)ₙ→CF≡CH (fluoroacetylene or fluoroethyne)+MFₙ+H2O
(ii)-d m CHF2CH2OH (2,2-difluoroethanol)+M(OH)ₙ→dimer/trimer/oligomer+MFₙ+H2O

Side reactions may also occur in Step (ii) through reactions with feed impurities. Example side product reactions from feed impurities are shown below, where M is K, Na, or Ca, and n is 1 or 2:
(ii)-e CF2ClCH2F (HCFC-133b)+M(OH)ₙ→CF₂═CHF (R-1123)+MClₙ+H2O
(ii)-f CF2ClCH2F (HCFC-133b)+M(OH)ₙ→CFCl═CHF (R-1122)+MFₙ+H2O
(ii)-g CHFClCHF2 (HCFC-133)+M(OH)ₙ→CHF═CF₂ (R-1123)+MClₙ+H2O
(ii)-h CHFClCHF2 (HCFC-133)+M(OH)ₙ→CFCl═CHF/CF2=CHCl (R-1122/R-1122a)+MFₙ+H2O
(ii)-i CF2ClCHFCl (HCFC-123a)+M(OH)ₙ→CF2=CFCl (R-1113)+MClₙ+H2O
(ii)-j CF2ClCHFCl (HCFC-123a)+M(OH)ₙ→CFCl═CFCl R-1112)+MFₙ+H2O
(ii)-k CHFClCH2F (HCFC-142b)+M(OH)ₙ→CHCl═CHF/CFCl═CH₂ (R-1131E/R-1131Z/R-1131a)+MFₙ+H2O
   (ii)-l CH3CHF2 (HFC-152a)+M(OH)ₙ→CH2=CHF (R-1141)+MFₙ+H2O
(ii)-m CHCl═CHF (HCFO-1131E/HCFC-1131Z)/CFCl═CH2 (HCFO-1131a)+M(OH)ₙ→CCl≡CH (chloroacetylene)+MFₙ+H2O

The reactor product stream 116 also comprises an amount of water which is removed in subsequent post processing steps as will be discussed herein. Additionally, minor amounts of higher-boiling organic byproducts, including difluoroethanols and oligomeric species such as dimers or trimers, may also be formed.

### Step (ii) Product Stream Composition

For the liquid phase dehydrofluorination reaction of Step (ii), the amount of HFO-1132 isomers in the second product stream 116 from the second reactor 114 is at least 30 mol %, at least 35 mol %, and for each of the foregoing, less than or equal to 100 mol %, for example, based on total moles of organic components of the composition.

For example, for a liquid phase dehydrofluorination reaction of Step (ii), referring to FIG. 1, in the second product stream 116 from the second reactor 114, the amount of HFO-1132 isomers may be at least 20 mol % and less than or equal to 45 mol %, the amount of HFC-143 may be at least 50 mol % and less than or equal to 75 mol %, and the combined amount of others (e.g., unreacted HCFC-133 isomers, HFC-152a, CTFE, HFO-1123, and HFO-1132a) is less than or equal to 5 mol %, based on total moles of organic components of the composition.

Exemplary compositions for the second product stream 116 from the second reactor 114 are set forth below in Table 16. The numerical ranges set forth in Table 16 below are understood to be prefaced by “about”.

TABLE 16
Composition of Second Product Stream 116

| | | | Combined amount of others |
|---|---|---|---|
| | | | (e.g., unreacted HCFC-133 |
| | | | isomers, HFC-152a, CTFE, |
| | | R-1132 | HFO-1123, and HFO-1132a) |
| Comp. | R-143 | (E/Z) | in the composition |
| 1 | 60-75 wt % | 20-35 wt % | 0-5 wt % |
| 2 | 55-75 wt % | 20-40 wt % | 0-5 wt % |
| 3 | 50-75 wt % | 20-45 wt % | 0-5 wt % |

The waste stream 138 may include a salt by-product of the dehydrohalogenating agent from the dehydrohalogenation reaction. The salt by-product may be isolated from the waste stream 138 using known techniques and converted back to the dehydrohalogenating agent using known methods. It may then be recycled to the dehydrohalogenation reaction independently for the step (ii) reaction (e.g., reactor 114), or with one or more of the organics provided herein. Specifically, referring to FIG. 1, the salt by-product and optionally the unreacted dehydrohalogenating agent in the waste stream 138 may be separated from organics in the waste stream 138 via a separator (not shown) before the salt by-product is converted back to the dehydrohalogenating agent and then returned to reactor 114. Alternatively, the dehydrohalogenating agent (resulting from the conversion of the salt by-product and the unreacted dehydrohalogenating agent) in the waste stream 138 may be separated from organics in the waste stream 138 after the salt by-product is converted back to the dehydrohalogenating agent and before the waste stream 138 enters a separator.

By way of non-limiting example, one such salt by-product may be potassium fluoride (KF), which is formed when potassium hydroxide (KOH) is used as the dehydrohalogenation agent. KF may be converted to KOH by treatment with Ca(OH)₂ (calcium hydroxide) according to the reaction: 2KF+Ca(OH)₂→2KOH+CaF₂.

The resulting KOH can be, optionally, concentrated and recycled back to the dehydrohalogenation reaction independently, or otherwise with one or more organics, as provided herein. CaF₂ will precipitate from the foregoing reaction while KOH is isolated and recycled back to reactor. Recycling of dehydrohalogenating agent leads to better efficiency of the reagent use. Moreover, the use of recycling of the by-product salt reduces dehydrohalogenating agent use, reduces costs of reagents and costs associated with disposal of the salt, and/or purchase of new raw material.

Reactor 114 or a plurality of reactors 114 may be driven in a primarily continuous, semi-batch, or batch process. While the Step (ii) reaction is underway, reaction products may be continuously or intermittently removed from reactor 114 into product stream 116. The reactor 114 may be cooled and depressurized to remove additional unreacted HFC-143 and any remaining reaction products into product stream 116. The reactor may be cooled to a temperature of less than about 150 °C, about 125 °C, about 120 °C, about 110 °C, about 100 °C, about 90 °C, about 80 °C, about 75 °C, about 70 °C, about 60 °C, about 50 °C, or within any range encompassed by any two of the foregoing values as endpoints, for example from about 60 °C to about 150 °C, preferably from about 60 °C to about 110 °C.

Once reactor 114 is cooled, the remaining material in the reactor may comprise at least one of water, MFₙ (where M=K, Na, or Ca and n=1 or 2), unreacted base, dimers/trimers/oligomers, difluoroethanol, small amounts of dissolved unreacted R-143, R-1132 isomers, R-1132a (vinylidene fluoride (VDF)), R-1123, R-1113, R-1112, CFCl═CHF (R-1122a), CF₂═CHCl (R-1122), CHCl═CHF (R-1131), CF≡CH (fluoroacetylene), and CCl≡CH (chloroacetylene). The remaining material may be purged as a waste stream 138. The waste stream may not be purged as the reactor 114 is running, so this process may be described as semi-batch or batch. Alternatively, the remaining material may be reused with fresh base from base feed stream 113.

### Step (ii) Post Processing

Referring again to FIG. 1, the product stream 116 from reactor 114 enters a reactor distillation column 133 to separate out unreacted HFC-143 and water for recycle in Stream 119. Stream 119 generally includes at least HFC-143 and water and is returned to the reactor 114 as reflux. The top stream 131 from reactor distillation column 133 includes primarily HFO-1132 isomers, and may include a number of other components such as, for example, at least one of R-1132a, CF≡CH (fluoroacetylene), R-1123, R-1122 (CFCl═CHF), R-1113 (CF2=CHCl), R-1112 (CHCl═CHF), CCl≡CH (chloroacetylene), unreacted R-143, and water. During Step (ii), volatile reaction products may be withdrawn from the reaction zone through the reactor distillation column, while unreacted HFC-143 and condensed water are returned to the reactor as reflux.

Stream 131 is sent to a dryer 140 to remove water that may be present in stream 131. The dryer 140 is packed with a drying agent which may comprise at least one of 3 A molecular sieves, 4 A molecular sieves, 5 A molecular sieves, alumina, CaSO4, and CaCl2. The dryer 140 will be periodically regenerated as is known in the art. The dryer may be periodically regenerated by purging with heated inert gas, followed by cooling prior to reuse. For example, dryer 140 may be regenerated when the dryer is no longer effective at adsorbing water. Regeneration may be accomplished by feeding hot nitrogen gas into the dryer 140 to drive off water. The dryer 140 may then be cooled with nitrogen at ambient temperature to prepare for the next drying cycle.

Once stream 131 is dried, dried product stream 132 enters first distillation column 118 to remove lights generated in the second reactor 114 through lights stream 120. Lights stream 120 may include, for example, at least one of R-1132E, R-1132a, R-1123, R-1113 and CF≡CH. Lights stream 120 may be vented, collected, oxidized, flared, or otherwise disposed of as is known in the art. The bottoms stream 122 comprises mostly R-143 and R-1132Z, and may comprise additional side products including, for example, at least one of R-1112, R-1122a, R-1122, R-1131, (CCl≡CH).

Suitable column conditions for the first distillation column 118 are as follows.

The number of stages for the first distillation column 118 may be as low as about 20, about 22, about 24, about 26, about 28, or as high as about 32, about 34, about 36, about 38, about 40, about 50, about 60, about 70, about 80, about 90, about 100 or within any range encompassed by any two of the foregoing values as endpoints, for example, from about 20 to about 100.

The temperature within the first distillation column 118 may be as low as about −20 °C, about 0 °C, about 20 °C, about 40 °C, about 60 °C, about 80 °C, about 100 °C, or as high as about 120 °C, about 140 °C, about 160 °C, about 180 °C, about 200 °C, or within any range encompassed by two of the foregoing values as endpoints. The temperature may be from about −20 °C to about 200 °C.

The pressure within the first distillation column 118 may be as low as about 100 torr, about 300 torr, about 600 torr, about 0 psig, about 20 psig, about 40 psig, about 60 psig, or as high as about 80 psig, about 100 psig, about 120 psig, about 140 psig, about 160 psig, about 180 psig, about 200 psig, about 250 psig, about 300 psig, about 350 psig or any range using any of the foregoing values as endpoints. The pressure may preferably be from about 0 psig to about 350 psig.

Bottom stream 122 from first distillation column 118 enters second distillation column 124 to separate HFO-1132Z from unreacted HFC-143. The top stream 126 from the second distillation column 124 contains HFO-1132 Z with a purity of, for example, of as little as about 90 wt. %, about 95 wt. %, or as much as about 99.0 wt. %, about 99.5 wt. %, about 99.9 wt. %, or within any value encompassed by any of the foregoing values as endpoints, such as from about 90 wt. % to about 99.9 wt. %, or about 95 wt. % to about 99.9 wt. %, where weight percent is calculated as based upon the total weight of stream 126. The bottom stream 128 from the second distillation column 124 contains HFC-143 with a purity of, for example, at least about 80 mol %, about 90 mol %, about 95 mol %, about 99.0 mol %, about 99.5 mol %, about 99.9 mol %, or within any range encompassed by any of the foregoing values as endpoints, such as about 80 mol % to 99.9 mol %, where mol % is calculated based on the total moles of organic components of the bottom stream 128. The bottoms stream 128 also comprises side products including, for example, at least one of R-1112, R-1122a, R-1122, R-1131, CCl≡CH. Suitable column conditions for the second distillation column 124 are as follows.

The number of stages for the second distillation column 124 may be as low as about 20, about 22, about 24, about 26, about 28, or as high as about 32, about 34, about 36, about 38, about 40, about 50, about 60, about 70, about 80, about 90, about 100 or within any range encompassed by any two of the foregoing values as endpoints, for example, from about 20 to about 100.

The temperature within the second distillation column 124 may be as low as about −20 °C, about 0 °C, about 20 °C, about 40 °C, about 60 °C, about 80 °C, about 100 °C, or as high as about 120 °C, about 140 °C, about 160 °C, about 180 °C, about 200 °C, or within any range encompassed by two of the foregoing values as endpoints. The temperature may be from about 0 °C to about 200 °C.

The pressure within the second distillation column 124 may be as low as about 100 torr, about 300 torr, about 600 torr, about 0 psig, about 20 psig, about 40 psig, about 60 psig, or as high as about 80 psig, about 100 psig, about 120 psig, about 140 psig, about 160 psig, about 180 psig, about 200 psig, about 250 psig, about 300 psig, about 350 psig, or any range using any of the foregoing values as endpoints. The pressure may \ be from about 0 psig to about 350 psig.

The top stream 126 from the second distillation column 124 may be collected and stored and/or may be used as a feedstock for a subsequent 1132 reaction process. In a preferred example, the top stream 126 comprising primarily high purity (e.g., 95 wt. % or higher) HFO-1132Z is used as the feedstock for the step (iii) isomerization process for converting the HFO-1132Z into HFO-1132E.

The bottom stream 128 from the second distillation column 124 enters third distillation column 130 to separate R-143 from side products and impurities. The top stream 136 from the third distillation column 130 contains primarily HFC-143 with a purity of, for example, at least about 80 mol %, about 90 mol %, about 95 mol %, about 99.0 mol %, about 99.5 mol %, about 99.9 mol %, or as great as about 100 mol %, or within any range encompassed by any of the foregoing values as endpoints, such as about 80 mol % to about 100 mol %, wherein mol % is calculated based on the total amount of organic components of the top stream 136. The top stream 136 is recycled and sent back to reactor 114 as recovered R-143. The bottoms stream 134 from third distillation column 130 comprises side products and impurities including, for example, at least one of R-1112, R-1122a, R-1122, R-1131, CCl≡CH, and may comprise a small amount of R-143.

For the liquid phase dehydrofluorination reaction of Step (ii), referring to FIG. 1, the combined amount or purity of HFO-1132 isomers (e.g., HFO-1132Z, HFO-1132E, and HFO-1132a) in the top stream 120 from first distillation column 118 may be at least 45 mol %, at least 50 mol %, at least 55 mol %, or at least 60 mol %, and for each of the foregoing, less than or equal to 100 mol %, for example, based on total moles of organic components in the composition.

For the liquid phase dehydrofluorination reaction of Step (ii), referring to FIG. 1, the amount of HFO-1123 in the top stream 120 from first distillation column 118 may be less than 0.5 mol %, less than 1 mol %, less than 2 mol %, less than 5 mol %, or at least 10 mol %, at least 15 mol %, or at least 20 mol %, and for each of the foregoing, less than or equal to 100 mol %, for example, based on total moles of organic components in the composition. The HFO-1123 separated from the product stream 116 may be further recycled and hydrogenated to produce HFC-143.

For the liquid phase dehydrofluorination reaction of Step (ii), referring to FIG. 1, the amount of HFO-1132Z in the bottom stream 122 from first distillation column 118 may be at least 15 mol %, at least 20 mol %, at least 25 mol %, at least 30 mol %, at least 35 mol %, and for each of the foregoing, less than or equal to 100 mol %, for example, based on total moles of organic components in the composition.

For the liquid phase dehydrofluorination reaction of Step (ii), referring to FIG. 1, the amount of HFO-1132Z in the top stream 126 (which may be referred to as a reactant composition for a further reaction, or a product composition from the described process) from the second distillation column 124 may be at least 95 wt. %, at least 96 wt. %, at least 97 wt. %, at least 95 wt. %, or at least 99 wt. %, and for each of the foregoing, less than or equal to 100 wt. %, for example, based on total moles of organic components in the composition.

Top stream 126 may comprise 1,1,1-trifluoroethane (HFC-143a) and trifluoroethylene (HFO-1123), if present, in a combined amount of less than 2000 ppm, less than 1500 ppm, less than 1000 ppm, less than 500 ppm, less than 100 ppm, less than 50 ppm, or less than 20 ppm, and for each of the foregoing, greater than or equal to 0 ppm, for example, based on total weight of organic components of the reactant composition.

The amount of 1,1,2-trifluoroethane (HFC-143) in the reactant composition, if present, may be less than 1.00 wt. %, less than 0.75 wt. %, less 0.50 wt. %, less than 0.10%, less than 0.01 wt. %, or less than 0.001 wt. %, as based on total weight of organic components of the reactant composition.

The amount of 1,1-difluoroethane (HFC-152a) in the reactant composition, if present, may be less than 1.00 wt. %, less than 0.75 wt. %, or less than 0.50 wt. %, less than 0.10%, less than 0.01 wt. %, or less than 0.001 wt. %, as based on total weight of organic components of the reactant composition.

For the liquid phase dehydrofluorination reaction of Step (ii), referring to FIG. 1, the amount of HFC-143 in the bottom stream 128 from the second distillation column 124 may be at least 95 mol %, at least 96 mol %, at least 97 mol %, at least 95 mol %, or at least 99 mol %, and for each of the foregoing, less than or equal to 100 mol %, for example, based on total moles of organic components in the composition.

### VI. Step (iii)

### General Process

The 1,2-difluoroethylene (HFO-1132) obtained in Step (ii) above may be produced as a mixture containing both the trans-1,2-difluoroethylene (HFO-1132E) and cis-1,2-difluoroethylene (HFO-1132Z) isomers. Step (iii) may be an isomerization of HFO-1132Z into HFO-1132E. Top stream 126 may be used as a reactant composition for step (iii).

The isomerization of HFO-1132Z may take place in the presence of an optional catalyst, such as reactive monomers like chlorotrifluoroethylene (CTFE); alumina (Al2O3); chromia (Cr2O3); zeolites; fluorinated metal oxides; basic catalysts; and halide-promoted catalysts. An amount of catalyst may be added to the isomerization reaction in an amount from as little as 1 wt. %, 2 wt. %, 4 wt. %, 6 wt. %, 8 wt. %, or 10 wt. % to as great as 12 wt. %, 13 wt. %, 16 wt. %, 18 wt. %, or 20 wt. %, or any range using any two of the foregoing values as endpoints, such as 1 wt. % to 20 wt. %, 2 wt. % to 18 wt. %, 4 wt. % to 16 wt. %, 6 wt. % to 13 wt. %, 8 wt. % to 12 wt. %, or 10 wt. % to 12 wt. %, based on total weight of organic components of the reactant composition.

Further, an optional diluent may be added to the isomerization reaction. Suitable diluents may comprise nitrogen gas or argon gas.

In step (iii), the cis-1,2-difluoroethylene (HFO-1132Z) isomer may be converted to the trans-1,2-difluoroethylene (HFO-1132E) isomer either by exposure to heat and/or a catalyst to yield a crude product composition comprising at least 5 wt. % trans-1,2-difluoroethylene (HFO-1232E), at least 10 wt. % trans-1,2-difluoroethylene (HFO-1232E), at least 18 wt. % trans-1,2-difluoroethylene (HFO-1232E), at least 20 wt. % trans-1,2-difluoroethylene (HFO-1232E), at least 30 wt. % trans-1,2-difluoroethylene (HFO-1232E), or greater.

Crude product composition 126 may comprise an amount of cis-1,2-difluoroethylene (HFO-1132Z) from as little as about 10 wt. %, about 15 wt. %, about 20 wt. %, about 25 wt. %, or about 30 wt. % to as great as about 35 wt. %, about 40 wt. %, about 50 wt. %, about 60 wt. %, about 66 wt. %, about 70 wt. %, or any range using any two of the forgoing values as endpoints, such as from about 10 wt. % to 70 wt. %, from about 15 wt. % to 66 wt. %, from about 20 wt. % to 60 wt. % from about 25 wt. % to 50 wt. %, from about 30 wt. % to 40 wt. %, or from about 30 wt. % to 35 wt. %, based on the total weight of the organic components of the crude product composition.

The amount of 1,1,1-trifluoroethane (HFC-143a) and trifluoroethylene (HFO-1123) in the crude product composition, if present, in a combined amount of less than 2000 ppm, less than 1500 ppm, less than 1000 ppm, less than 700 ppm, less than 500 ppm, less than 100 ppm, less than 50 ppm, or less than 20 ppm, and for each of the foregoing, greater than or equal to 0 ppm, for example, based on total weight of organic components of the reactant composition.

The amount of 1,1,2-trifluoroethane (HFC-143) in the crude product composition, if present, may be less than 1.00 wt. %, less than 0.88 wt. %, less than 0.75 wt. %, or less than 0.50 wt. %, and for each of the foregoing, greater than or equal to 0.01 wt. %, for example, based on total weight of organic components of the reactant composition.

The amount of 1,1-difluoroethane (HFC-152a) in the crude product composition, if present, may be less than 1.50 wt. %, less than 1.00 wt. %, less than 0.88 wt. %, less than 0.75 wt. %, or less than 0.50 wt. %, and for each of the foregoing, greater than or equal to 0.01 wt. %, for example, based on total weight of organic components of the reactant composition.

The crude product composition from isomerization reactor can be further distilled to yield a purified product composition comprising, consisting essentially of, or consisting of, the trans-1,2-difluoroethylene (HFO-1132E) isomer in high purity, for example at least about 95 wt. %, at least about 99.0 wt. %, at least about 99.9 wt. %, at least about 99.99 wt. % or greater, based on total weight of organic components of the purified product composition.

The isomerization reaction may be conducted in any suitable reaction vessel or reactor, but it should preferably be constructed from materials which are resistant to corrosion including but not limited to nickel and its alloys, including Hastelloy (for example, Hastelloy C276), Inconel (for example Inconel 600), and Monel wherein the vessels which may be lined with fluoropolymers. These may be single pipe or multiple tubes packed with an isomerization catalyst.

### Reaction Conditions—Temperature

The temperature range for the isomerization reaction may be as low as about 100 °C, about 150 °C, about 200 °C, about 250 °C, about 300 °C, about 350 °C, about 400 °C, or as high as about 490 °C, about 550 °C, about 550 °C, about 600 °C, about 650 °C, about 700 °C, about 750 °C, about 800 °C or within any range encompassed by two of the foregoing values as endpoints, for example from about 100 °C to about 800 °C, from about 150 °C to about 750 °C, from about 200 °C to about 700 °C, from about 250 °C to about 650 °C, from about 300 °C to about 600 °C, from about 350 °C to about 550 °C, or from about 400 °C to about 500 °C.

### Reaction Conditions—Pressure

The reaction may be conducted at atmospheric pressure, super-atmospheric pressure or under vacuum. The vacuum pressure can be from about 5 psig, about 10 psig, about 25 psig, about 50 psig, about 100 psig, about 150 tpsigorr, about 200 psig, about 250 psig, or any range using any of the foregoing values as endpoints, for example from about 5 psig to about 250 psig, about 10 psig to about 200 psig, about 25 psig to about 150 psig, or about 50 psig to about 100 psig.

### Reaction Conditions—Contact Time

Contact time of the reactants with the catalyst may range from about 0.5 seconds, about 1 second, about 5 seconds, about 10 seconds, to about 20 seconds, about 30 seconds, about 60 seconds, or about 120 seconds, or any range using any of the foregoing values as endpoints, for example from about 0.5 seconds to about 120 seconds, about 1 second to about 60 seconds, about 5 seconds to about 30 seconds, or about 10 seconds to about 20 seconds. However, longer or shorter times can be used. Shorter contact times yield lower amounts of impurities and longer contact times at lower temperatures may also yield lower amounts of impurities.

### Reaction Conditions—Environment

The reaction may also be conducted in an inert atmosphere substantially in the absence of oxygen. For example, the amount of oxygen present during the reaction may be less than 15 wt. %, less than 10 wt. %, less than 5 wt. %, or less than 1 wt. %, or any range using any two of the foregoing values as endpoints, for example from about 1 wt. % to about 15 wt. %, or about 5 wt. % to about 10 wt. %, based on a total weight of the reactants in the reactor.

The reaction may also be conducted substantially in the absence of water. For example, the amount of water present during the reaction may be less than 5 wt. %, less than 1 wt. %, less than 0.5 wt. %, or less than 0.05 wt. %, or any range using any two of the foregoing values as endpoints, for example 0.05 wt. % to about 5 wt. %, or about 0.5 wt. % to about 1 wt. %, based on a total weight of the reactants in the reactor.

### EXAMPLES

### Example 1

### Dehydrochlorination Reaction of Step (i) for Feed Purification

The experimental apparatus used includes a 2-gallon Hastelloy C pressure vessel (series 4660) and an Eldex pump used to feed KOH solution. Reaction was performed in batch mode. The reactor was loaded with the 2545 grams of organic feed first, heated to 108 °C under stirring (600 rpm), and then 3370 g of 45% KOH solution was slowly added over three hours. After complete addition of KOH solution, the reaction mixture was kept at 108 °C under stirring for an additional 5 hours. At the end, the reactor temperature was reduced to 70 °C and the reactor content was opened to a dry ice cooled product collection cylinder (PCC). About 1959 g of the organic product was collected in the PCC. The overall mass balance was 98.6%.

The organic feed and product were analyzed using gas chromatography, results are shown in Table 17. It is assumed that the GC-FID area % are equal to weight %, and conversion and selectivity values were calculated using the equations shown above. Results are presented in Table 18.

Unaccounted selectivity for each reaction shows selectivity to “heavies” that are left in the Parr reactor as undesired byproducts (waste). For example, for entry 1 (Example 1), R123a reaction showed 96.5% conversion, 7.6% selectivity to R1113, and therefore 92.4% selectivity to heavy undesired byproducts. R133/R133b reactions showed 84.6% conversion, 72.0% selectivity to R1123, and therefore 28.0% selectivity to heavy undesired byproducts. R143 reaction showed 10.6% conversion, 18.3% selectivity to R1132 isomers, and therefore 81.7% selectivity to heavy undesired byproducts.

TABLE 17
GC-FID area % of the organic feed and product

| Molecule | Feed | Product |
|---|---|---|
| R143 | 69.99 | 81.32 |
| R133b | 13.54 | 3.24 |
| R133 | 2.66 | 0.26 |
| R123a | 10.20 | 0.47 |
| R152a | 2.27 | 2.49 |
| R142 | 0.54 | 0.04 |
| R160 | 0.09 | 0.00 |
| R1113 | 0.00 | 0.74 |
| R1123 | 0.03 | 8.83 |
| R1132Z | 0.00 | 0.99 |
| R1132E | 0.00 | 0.07 |
| R1132a | 0.00 | 0.28 |
| Other | 0.68 | 1.29 |

TABLE 18
Reaction conditions and the corresponding conversion/selectivity values

| | | | R123a rxn | | R133b and R133 rxns | | R143 Rxn | |
|---|---|---|---|---|---|---|---|---|
| | KOH/ | | | Selec. | | Selec. | | Selec. |
| | org. | | | to | | to | | to |
| Temp | mole | KOH | Conv. | R1113 | Conv. | R1123 | Conv. | R1132 |
| °C | ratio* | Wt % | (%) | (%) | (%) | (%) | (%) | (%) |
| 108 | 1.0 | 45 | 96.5 | 7.6 | 84.6 | 72.0 | 10.6 | 18.3 |
| | (5.2) | | | | | | | |

*n(KOH)/n(R143 + R133b + R133 + 123a) mole ratio. The numbers in the parentheses show the n(KOH)/n(R133b + R133 + 123a) mole ratio.

### Example 2

### Dehydrochlorination Reaction of Step (i) for Feed Purification

The experimental apparatus used includes a 2-gallon Hastelloy C pressure vessel (series 4660) and an Eldex pump used to feed KOH solution. Reaction was performed in batch mode. The reactor was loaded with the 1790 grams of organic feed first, heated to 104 °C under stirring (600 rpm), and then 4070 g of 25% KOH solution was slowly added over three hours. After complete addition of KOH solution, the reaction mixture was kept at 104 °C under stirring for an additional 5 hours. At the end, the reactor temperature was reduced to 70 °C and the reactor content was opened to a dry ice cooled product collection cylinder (PCC). About 1290 g of the organic product was collected in the PCC. The overall mass balance was 98.7%.

The organic feed and product were analyzed using gas chromatography, results are shown in Table 19. It is assumed that the GC-FID area % are equal to weight %, and conversion and selectivity values were calculated using the equations shown above. Results are presented in Table 20.

TABLE 19
GC-FID area % of the organic feed and product

| Molecule | Feed | Product |
|---|---|---|
| R143 | 69.17 | 82.32 |
| R133b | 14.50 | 1.92 |
| R133 | 2.04 | 0.14 |
| R123a | 7.66 | 0.18 |
| R152a | 4.03 | 4.98 |
| R142 | 0.70 | 0.00 |
| R160 | 0.17 | 0.00 |
| R1113 | 0.13 | 0.69 |
| R1123 | 0.66 | 6.64 |
| R1132Z | 0.00 | 0.70 |
| R1132E | 0.00 | 0.00 |
| R1132a | 0.00 | 0.12 |
| Other | 0.94 | 2.32 |

TABLE 20
Reaction conditions and the corresponding conversion/selectivity values

| | | | R123a rxn | | R133b and R133 rxns | | R143 Rxn | |
|---|---|---|---|---|---|---|---|---|
| | KOH/ | | | Selec. | | Selec. | | Selec. |
| | org. | | | to | | to | | to |
| Temp | mole | KOH | Conv. | R1113 | Conv. | R1123 | Conv. | R1132 |
| °C | ratio* | Wt % | (%) | (%) | (%) | (%) | (%) | (%) |
| 104 | 1.0 | 25 | 98.3 | 6.3 | 91.6 | 39.4 | 14.2 | 7.8 |
| | (5.3) | | | | | | | |

*n(KOH)/n(R143 + R133b + R133 + 123a) mole ratio. The numbers in the parentheses show the n(KOH)/n(R133b + R133 + 123a) mole ratio.

### Example 3

### Dehydrochlorination Reaction of Step (i) for Feed Purification

The experimental apparatus used includes a 2-gallon Hastelloy C pressure vessel (series 4660) and an Eldex pump used to feed KOH solution. Reaction was performed in batch mode. The reactor was loaded with 890 grams of organic feed first, heated to 104 °C under stirring (600 rpm), and then 5060 g of 10 wt % KOH solution was slowly added over three hours. After complete addition of KOH solution, the reaction mixture was kept at 107 °C under stirring for an additional 5 hours. At the end, the reactor temperature was reduced to 70 °C and the reactor content was opened to a dry ice cooled product collection cylinder (PCC). About 678 g of the organic product was collected in the PCC. The overall mass balance was 99.3%.

The organic feed and product were analyzed using gas chromatography, results are shown in Table 21. It is assumed that the GC-FID area % are equal to weight %, and conversion and selectivity values were calculated using the equations shown above. Results are presented in Table 22.

TABLE 21
GC-FID area % of the organic feed and product

| Molecule | Feed | Product |
|---|---|---|
| R143 | 69.18 | 80.87 |
| R133b | 14.50 | 2.01 |
| R133 | 2.05 | 0.42 |
| R123a | 7.66 | 0.23 |
| R152a | 4.03 | 4.67 |
| R142 | 0.70 | 0.00 |
| R160 | 0.17 | 0.00 |
| R1113 | 0.13 | 1.25 |
| R1123 | 0.66 | 8.28 |
| R1132Z | 0.00 | 0.28 |
| R1132E | 0.00 | 0.00 |
| R1132a | 0.00 | 0.07 |
| Other | 0.92 | 1.93 |

TABLE 22
Reaction conditions and the corresponding conversion/selectivity values

| | | | R123a rxn | | R133b and R133 rxns | | R143 Rxn | |
|---|---|---|---|---|---|---|---|---|
| | KOH/ | | | Selec. | | Selec. | | Selec. |
| | org. | | | to | | to | | to |
| Temp | mole | KOH | Conv. | R1113 | Conv. | R1123 | Conv. | R1132 |
| °C | ratio* | Wt % | (%) | (%) | (%) | (%) | (%) | (%) |
| 107 | 1.0 | 10 | 97.7 | 14.4 | 90.7 | 55.2 | 10.9 | 4.6 |
| | (5.3) | | | | | | | |

*n(KOH)/n(R143 + R133b + R133 + 123a) mole ratio. The numbers in the parentheses show the n(KOH)/n(R133b + R133 + 123a) mole ratio.

### Example 4

### Dehydrochlorination Reaction of Step (i) for Feed Purification

The experimental apparatus used includes a 2-gallon Hastelloy C pressure vessel (series 4660) and an Eldex pump used to feed KOH solution. Reaction was performed in batch mode. The reactor was loaded with the 2828 grams of organic feed first, heated to 104 °C under stirring (600 rpm), and then 640 g of 45 wt % KOH solution was slowly added over three hours. After complete addition of KOH solution, the reaction mixture was kept at 104 °C under stirring for an additional 5 hours. At the end, the reactor temperature was reduced to 70 °C and the reactor content was opened to a dry ice cooled product collection cylinder (PCC). About 2668 g of the organic product was collected in the PCC. The overall mass balance was 93.8%.

The organic feed and product were analyzed using gas chromatography, results are shown in Table 23. It is assumed that the GC-FID area % are equal to weight %, and conversion and selectivity values were calculated using the equations shown above. Results are presented in Table 24.

TABLE 23
GC-FID area % of the organic feed and product

| Molecule | Feed | Product |
|---|---|---|
| R143 | 70.46 | 71.78 |
| R133b | 14.11 | 13.54 |
| R133 | 1.83 | 1.65 |
| R123a | 7.25 | 0.09 |
| R152a | 3.97 | 4.06 |
| R142 | 0.81 | 0.53 |
| R160 | 0.15 | 0.14 |
| R1113 | 0.10 | 5.19 |
| R1123 | 0.34 | 1.54 |
| R1132Z | 0.00 | 0.00 |
| R1132E | 0.00 | 0.00 |
| R1132a | 0.00 | 0.00 |
| Other | 0.98 | 1.51 |

TABLE 24
Reaction conditions and the corresponding conversion/selectivity values

| | | | R123a rxn | | R133b and R133 rxns | | R143 Rxn | |
|---|---|---|---|---|---|---|---|---|
| | KOH/ | | | Selec. | | Selec. | | Selec. |
| | org. | | | to | | to | | to |
| Temp | mole | KOH | Conv. | R1113 | Conv. | R1123 | Conv. | R1132 |
| °C | ratio* | Wt % | (%) | (%) | (%) | (%) | (%) | (%) |
| 104 | 0.2 | 45 | 98.8 | 87.9 | 19.9 | 98.5 | 3.9 | 0.0 |
| | (1.0) | | | | | | | |

*n(KOH)/n(R143 + R133b + R133 + 123a) mole ratio. The numbers in the parentheses show the n(KOH)/n(R133b + R133 + 123a) mole ratio.

### Example 5

### Dehydrochlorination Reaction of Step (i) for Feed Purification

The experimental apparatus used includes a 2-gallon Hastelloy C pressure vessel (series 4660) and an Eldex pump used to feed KOH solution. Reaction was performed in batch mode. Reactor was loaded with 2839 grams of organic feed first, heated to 112 °C under stirring (600 rpm), and then 1840 g of 45 wt % KOH solution was slowly added over three hours. After complete addition of KOH solution, the reaction mixture was kept at 112 °C under stirring for an additional 5 hours. At the end, the reactor temperature was reduced to 70 °C and the reactor content was opened to a dry ice cooled product collection cylinder (PCC). About 1995 g of the organic product was collected in the PCC. The overall mass balance was 94.3%.

The organic feed and product were analyzed using gas chromatography, results are shown in Table 25. It is assumed that the GC-FID area % are equal to weight %, and conversion and selectivity values were calculated using the equations shown above. Results are presented in Table 26.

TABLE 25
GC-FID area % of the organic feed and product

| Molecule | Feed | Product |
|---|---|---|
| R143 | 68.46 | 76.83 |
| R133b | 13.71 | 8.36 |
| R133 | 1.76 | 0.60 |
| R123a | 6.92 | 0.00 |
| R152a | 4.18 | 4.77 |
| R142 | 0.83 | 0.11 |
| R160 | 0.15 | 0.09 |
| R1113 | 0.11 | 2.21 |
| R1123 | 2.08 | 4.76 |
| R1132Z | 0.00 | 0.34 |
| R1132E | 0.00 | 0.00 |
| R1132a | 0.00 | 0.16 |
| Other | 1.80 | 1.44 |

TABLE 26
Reaction conditions and the corresponding conversion/selectivity values

| | | | R123a rxn | | R133b and R133 rxns | | R143 Rxn | |
|---|---|---|---|---|---|---|---|---|
| | KOH/ | | | Selec. | | Selec. | | Selec. |
| | org. | | | to | | to | | to |
| Temp | mole | KOH | Conv. | R1113 | Conv. | R1123 | Conv. | R1132 |
| °C | ratio* | Wt % | (%) | (%) | (%) | (%) | (%) | (%) |
| 112 | 0.5 | 45 | 100 | 27.4 | 62.0 | 19.8 | 21.1 | 3.2 |
| | (3.0) | | | | | | | |

*n(KOH)/n(R143 + R133b + R133 + 123a) mole ratio. The numbers in the parentheses show the n(KOH)/n(R133b + R133 + 123a) mole ratio.

### Example 6

Table 27 shows the summary of Examples 1-5. Since the purpose of the Step (i) is increasing the R143 purity, in the optimum reaction conditions the R143 conversion is minimized while R123, R133, and R133b conversions are maximized. In addition, it is preferred to have higher selectivity to R1113 from R123a reaction and higher selectivity to R1123 from R133/R133b reactions. The R1113 and R1123 molecules could be easily separated from R143 to be recycled and/or hydrogenated in a separate reactor to produce R143.

TABLE 27
Comparison table of Examples 1-5

| | | | | | | R133b and | | | |
|---|---|---|---|---|---|---|---|---|---|
| | | | | R123a rxn | | R133 rxns | | R143 Rxn | |
| | | | | | Selec. | | Selec. | | Selec. |
| | | | | | to | | to | | to |
| | Temp | KOH/ | KOH | Conv. | R1113 | Conv. | R1123 | Conv. | R1132 |
| | °C | org.* | Wt % | (%) | (%) | (%) | (%) | (%) | (%) |
| Ex. | 108 | 1.0 | 45 | 96.5 | 7.6 | 84.6 | 72.0 | 10.6 | 18.3 |
| 1 | | (5.2) | | | | | | | |
| Ex. | 104 | 1.0 | 25 | 98.3 | 6.3 | 91.6 | 39.4 | 14.2 | 7.8 |
| 2 | | (5.3) | | | | | | | |
| Ex. | 107 | 1.0 | 10 | 97.7 | 14.4 | 90.7 | 55.2 | 10.9 | 4.6 |
| 3 | | (5.3) | | | | | | | |
| Ex. | 104 | 0.2 | 45 | 98.8 | 87.9 | 19.9 | 98.5 | 3.9 | 0.0 |
| 4 | | (1.0) | | | | | | | |
| Ex. | 112 | 0.5 | 45 | 100 | 27.4 | 62.0 | 19.8 | 21.1 | 3.2 |
| 5 | | (3.0) | | | | | | | |

*n(KOH)/n(R143 + R133b + R133 + 123a) mole ratio. The numbers in the parentheses show the n(KOH)/n(R133b + R133 + 123a) mole ratio.

### Example 7

### Dehydrochlorination Reaction of Step (i) Using Alternative Bases

Examples 1-5 are repeated with using the same reaction apparatus and conditions, but with alternative bases including: sodium hydroxide (NaOH), calcium oxide (CaO), and calcium hydroxide (Ca(OH)₂). Similar results are obtained.

### Comparative Example 8

### Dehydrofluorination Reaction of Step (ii)

The experimental setup used includes a 2-gallon Hastelloy C pressure vessel (Parr Instruments). The vessel is equipped with an agitator. The exit of reactor goes into distillation column (1″OD, 18″ length). The product of the distillation goes into product collection cylinder (PCC). Reaction was performed in the batch mode. Reactor was loaded with 1398 grams of organic feed. The reactor was heated to 116 °C with stirrer (600 rpm) on. When the reactor reached temperature and temperature was stable, 2200 g of 45% KOH solution was added over three hours using Eldex pump. After complete addition of KOH solution, the reaction mixture was kept at 116 °C under stirring for an additional 0.5 hours. Then the reactor exit was opened to distillation column. Due to some fluctuations in temperature, system was allowed to equilibrate for 2 hours (total reflux). Then the hold time of 10 hours was started. At the end of the hold time, the reactor temperature was reduced to 70 °C and the reactor was opened to a dry ice cooled product collection cylinder (PCC). About 897 g of the organic product was collected in the PCC. The overall mass balance was 97.7%.

The organic feed and product were analyzed using gas chromatography, results are shown in Table 28. It is assumed that the GC-FID area % are equal to weight %, and conversion and selectivity values were calculated using the equations shown above. Results are presented in Table 29.

TABLE 28
GC-FID area % of the organic feed and product

| Molecule | Feed | Product |
|---|---|---|
| R143 | 68.89 | 79.63 |
| R133s | 16.95 | 0.97 |
| R123a | 8.62 | 0.15 |
| R152a | 3.46 | 4.79 |
| R1113 | 0.15 | 1.39 |
| R1123 | 0.50 | 5.37 |
| R1132Z | 0 | 4.79 |
| R1132E | 0 | 0.37 |
| R1132a | 0 | 0.43 |
| Other | 1.9 | 2.12 |

TABLE 29
Reaction conditions and the corresponding
conversion/selectivity values

| | Mole | | | | | | | |
|---|---|---|---|---|---|---|---|---|
| | mole | | | | | | | |
| | ratio | | | | | | | |
| | of | | Conv | Selec. | Selec. | Selec. | Selec. | |
| | KOH | KOH | of | to | to | To | to | |
| T, | to | Weight | 143. | 1132Z | R1132E | 1132a | Heavies | Yield, |
| °C | Org* | %, % | (%) | (%) | (%) | (%) | (%) | %** |
| 116 | 1.5 | 45 | 26 | 23 | 2 | 2 | 74 | 6.3 |

*n(KOH)/n(R143 + R133b + R133 + 123a) mole ratio.
**Yield is defined as moles of 1132Z and 1132E made divided by initial amount of 143.

### Comparative Example 9

### Dehydrofluorination Reaction of Step (ii)

The experimental setup used includes a 2-gallon Hastelloy C pressure vessel (Parr Instruments). The vessel is equipped with an agitator. The exit of reactor goes into distillation column (1″OD, 18″ length). The product of the distillation goes into product collection cylinder (PCC). Reaction was performed in batch mode. The reactor was loaded with the 2200 grams of 45% KOH. The reactor was heated to 116 °C with stirrer (600 rpm) on. When the reactor reached temperature and temperature was stable, 1576 g of organic feed was added over three hours. After complete addition of organic, the reaction mixture was kept at 116 °C under stirring for an additional 0.5 hours. Then the reactor exit was opened to distillation column. Due to some fluctuations in temperature, system was allowed to equilibrate for 2 hours (total reflux). Then the hold time of 10 hours was started. At the end of the hold time, the reactor temperature was reduced to 70 °C and the reactor content was opened to a dry ice cooled product collection cylinder (PCC). About 1244 g of the organic product was collected in the PCC. The overall mass balance was 96.3%.

The organic feed and product were analyzed using gas chromatography, results are shown in Table 30. It is assumed that the GC-FID area % are equal to weight %, and conversion and selectivity values were calculated using the equations shown above. Results are presented in Table 31.

TABLE 30
GC-FID area % of the organic feed and product

| Molecule | Feed | Product |
|---|---|---|
| R143 | 70.23 | 72.76 |
| R133s | 16.47 | 3.62 |
| R123a | 8.89 | 0.07 |
| R152a | 2.64 | 3.12 |
| R1113 | 0 | 2.19 |
| R1123 | 0.36 | 11.57 |
| R1132Z | 0 | 4.26 |
| R1132E | 0 | 0.31 |
| R1132a | 0 | 0.65 |
| Other | 0 | 1.46 |

TABLE 31
Reaction conditions and the corresponding
conversion/selectivity values

| | Mole | | | | | | | |
|---|---|---|---|---|---|---|---|---|
| | mole | | | | | | | |
| | ratio | | | | | | | |
| | of | | Conv | Selec. | Selec. | Selec. | Selec. | |
| | KOH | KOH | of | to | to | To | to | |
| T, | to | Weight | 143. | 1132Z | R1132E | 1132a | Heavies | Yield, |
| °C | Org* | %, % | (%) | (%) | (%) | (%) | (%) | %** |
| 116 | 1.3 | 45 | 18 | 34 | 2 | 5 | 58 | 6.7 |

*n(KOH)/n(R143 + R133b + R133 + 123a) mole ratio.
**Yield is defined as moles of 1132Z and 1132E made divided by initial amount of 143.

### Example 10

### Dehydrofluorination Reaction of Step (ii)

The experimental setup used includes a 2-gallon Hastelloy C pressure vessel (Parr Instruments). The vessel is equipped with an agitator. The exit of reactor goes into distillation column (1″OD, 18″ length). The product of the distillation goes into product collection cylinder (PCC). Reaction was performed in batch mode. The reactor was loaded with the 2200 grams of 45% KOH. The reactor was heated to 121 °C with the stirrer on 600 rpm speed. When the reactor reached temperature and temperature was stable, 1125 g of organic feed was added over three hours. After complete addition of organic, the reaction mixture was kept at 121 °C under stirring for an additional 0.5 hours. Then the reactor exit was opened to distillation column. Due to some fluctuations in temperature, system was allowed to equilibrate for 2 hours (total reflux). Then the hold time of 10 hours was started. At the end of the hold time, the reactor temperature was reduced to 70 °C and the reactor content was opened to a dry ice cooled product collection cylinder (PCC). About 901 g of the organic product was collected in the PCC. The overall mass balance was 99.3%.

The organic feed and product were analyzed using gas chromatography, results are shown in Table 32. It is assumed that the GC-FID area % are equal to weight %, and conversion and selectivity values were calculated using the equations shown above. Results are presented in Table 33.

TABLE 32
GC-FID area % of the organic feed and product

| Molecule | Feed | Product |
|---|---|---|
| R143 | 95.4 | 74.04 |
| R133s | 2.40 | 0.28 |
| R123a | 0.20 | 0.00 |
| R152a | 0.11 | 0.15 |
| R1113 | 0.00 | 0.34 |
| R1123 | 0.01 | 0.26 |
| R1132Z | 0.00 | 21.73 |
| R1132E | 0.00 | 1.01 |
| R1132a | 0.00 | 0.49 |
| Other | 1.3 | 1.71 |

TABLE 33
Reaction conditions and the corresponding
conversion/selectivity values

| | Mole | | | | | | | |
|---|---|---|---|---|---|---|---|---|
| | mole | | | | | | | |
| | ratio | | | | | | | |
| | of | | Conv | Selec. | Selec. | Selec. | Selec. | |
| | KOH | KOH | of | to | to | To | to | |
| T, | to | Weight | 143. | 1132Z | R1132E | 1132a | Heavies | Yield, |
| °C | Org* | %, % | (%) | (%) | (%) | (%) | (%) | %** |
| 121 | 1.4 | 45 | 38 | 63 | 3 | 1 | 32 | 25.1 |

*n(KOH)/n(R143 + R133b + R133 + 123a) mole ratio.
**Yield is defined as moles of 1132Z and 1132E made divided by initial amount of 143.

### Example 11

### Dehydrofluorination Reaction of Step (ii)

The experimental setup used includes a 2-gallon Hastelloy C pressure vessel (Parr Instruments). The vessel is equipped with an agitator. The exit of reactor goes into distillation column (1″OD, 18″ length). The product of the distillation goes into product collection cylinder (PCC). Reaction was performed in batch mode. The reactor was loaded with the 1600 grams of organic feed. The reactor was heated to 116 °C with stirrer (600 rpm) on. When the reactor reached temperature and temperature was stable, 2640 g of 45% KOH solution was added over three hours using Eldex pump. After complete addition of KOH solution, the reaction mixture was kept at 116 °C under stirring for an additional 0.5 hours. Then the reactor exit was opened to distillation column. Due to some fluctuations in temperature, system was allowed to equilibrate for 2 hours (total reflux). Then the hold time of 10 hours was started. At the end of the hold time, the reactor temperature was reduced to 70 °C and the reactor content was opened to a dry ice cooled product collection cylinder (PCC). About 1317 g of the organic product was collected in the PCC. The overall mass balance was 98.7%.

The organic feed and product were analyzed using gas chromatography, results are shown in Table 34. It is assumed that the GC-FID area % are equal to weight %, and conversion and selectivity values were calculated using the equations shown above. Results are presented in Table 35.

TABLE 34
GC-FID area % of the organic feed and product

| Molecule | Feed | Product |
|---|---|---|
| R143 | 99.57 | 74.00 |
| R133s | 0.28 | 0.00 |
| R123a | 0.00 | 0.00 |
| R152a | 0.00 | 0.00 |
| R1113 | 0.00 | 0.00 |
| R1123 | 0.00 | 0.22 |
| R1132Z | 0.00 | 22.20 |
| R1132E | 0.00 | 1.40 |
| R1132a | 0.00 | 1.83 |
| Other | 0.16 | 0.34 |

TABLE 35
Reaction conditions and the corresponding
conversion/selectivity values

| | Mole | | | | | | | |
|---|---|---|---|---|---|---|---|---|
| | mole | | | | | | | |
| | ratio | | | | | | | |
| | of | | Conv | Selec. | Selec. | Selec. | Selec. | |
| | KOH | KOH | of | to | to | To | to | |
| T, | to | Weight | 143. | 1132Z | R1132E | 1132a | Heavies | Yield, |
| °C | Org* | %, % | (%) | (%) | (%) | (%) | (%) | %** |
| 116 | 1.1 | 45 | 39 | 62 | 4 | 5 | 29 | 25.6 |

*n(KOH)/n(R143 + R133b + R133 + 123a) mole ratio.
** Yield is defined as moles of 1132Z and 1132E made divided by initial amount of 143.

### Example 12

### Summary of Comparative Examples 8-9 and Examples 10-11

This example shows a summary of Comparative Examples 8-9 and Examples 10-11 in Table 36. The purity of material had effect on the selectivity/yield. Higher yields could be obtained using the feed that had higher purity. The least amount of heavies was formed using the feed that had higher purity. The use of very high purity feed for Step (ii) is recommended (>95%). Therefore, the reactant composition for Step (ii) should be purified in Step (i) to remove impurities.

TABLE 36
Comparison table of Comparative Examples 8-9 and Examples 10-11

| | | | Mole | | | | | | | |
|---|---|---|---|---|---|---|---|---|---|---|
| | | | mole | | | | | | | |
| | | | ratio | | | | | | | |
| | | | of | | Conv | Selec. | Selec. | Selec. | Selec. | |
| | % 143 | | KOH | KOH | of | to | to | To | to | |
| | in the | T, | to | Weight | 143. | 1132Z | R1132E | 1132a | Heavies | Yield, |
| Entry | Feed | °C | Org* | %, % | (%) | (%) | (%) | (%) | (%) | %** |
| Comp. | 68.9 | 116 | 1.5 | 45 | 26 | 23 | 2 | 2 | 74 | 6.3 |
| Ex. 8 | | | | | | | | | | |
| Comp. | 70.2 | 116 | 1.3 | 45 | 18 | 34 | 2 | 5 | 58 | 6.7 |
| Ex. 9 | | | | | | | | | | |
| Ex. 10 | 95.4 | 121 | 1.4 | 45 | 38 | 63 | 3 | 1 | 32 | 25.1 |
| Ex. 11 | 99.6 | 116 | 1.1 | 45 | 39 | 62 | 4 | 5 | 29 | 25.6 |

*n(KOH)/n(R143 + R133b + R133 + 123a) mole ratio.
**Yield is defined as moles of 1132Z and 1132E made divided by initial amount of 143.

### Example 13

### Dehydrofluorination Reaction of Step (ii) Using Alternative Bases

Examples 10 and 11 are repeated with using the same reaction apparatus and conditions, but with alternative bases including: sodium hydroxide (NaOH), calcium oxide (CaO), and calcium hydroxide (Ca(OH)₂). Similar results are obtained.

### Example 14

### Integrated Process

The first liquid phase reactor performance is described in Example 1. Product from several batches (see Example 1 for composition) may be combined before sending to the preconditioning distillation column to remove HFO-1123, CTFE, HFO-1132Z/E, HFO-1132a, and other low boiling point molecules. The preconditioning distillation column may have 30 stages. The composition of the top and bottom stream from the preconditioning distillation column is shown in Table 37. The top stream is recycled to further produce HFC-143, and the bottom stream is sent to the second liquid phase reactor of Step (ii).

TABLE 37
GC-FID area % top and bottom stream from
the preconditioning distillation column

| | 1^{st} | 1^{st} |
|---|---|---|
| | column, | column, |
| | top | bottom |
| Molecule | stream | stream |
| R143 | 11.08 | 95.25 |
| R133b | 0.46 | 3.79 |
| R133 | 0.00 | 0.31 |
| R123a | 0.00 | 0.56 |
| R152a | 16.31 | 0.00 |
| R142 | 0.00 | 0.05 |
| R160 | 0.00 | 0.00 |
| R1113 | 4.86 | 0.00 |
| R1123 | 58.44 | 0.00 |
| R1132Z | 6.53 | 0.00 |
| R1132E | 0.46 | 0.00 |
| R1132a | 1.85 | 0.00 |

1650 grams of the material from the bottom stream of the 1st distillation column is sent to an empty 2-gallon Hastelloy C pressure vessel (series 4660). The reactor is heated to 116 °C with stirring at 600 rpm. When the reactor reaches temperature and temperature is stable, 2652 g of 45% KOH solution is added over three hours using Eldex pump. After complete addition of KOH solution, the reaction mixture is kept at 116 °C under stirring for an additional 0.5 hours. Then the reactor exit is opened to distillation column. Due to some fluctuations in temperature, system is allowed to equilibrate for 2 hours (total reflux). Then the hold time of 10 hours is started. At the end of the hold time, the reactor temperature is reduced to 70 °C and the reactor content is opened to a dry ice cooled product collection cylinder (PCC). About 1290 g of the organic product is collected in the PCC. The overall mass balance is about 99%.

The organic feed and product are analyzed using gas chromatography (GC-FID), results are shown in Table 38. Product from several batches may be combined before being sent to the first distillation column.

TABLE 38
Weight percentages of the second reactor product as well as the top
and bottom stream from the second and third distillation columns

| | | 2^{nd} | 2^{nd} | 3^{rd} | 3rd |
|---|---|---|---|---|---|
| | 2^{nd} | column, | column, | column, | column, |
| | reactor | top | bottom | top | bottom |
| Molecule | product | stream | stream | stream | stream |
| R143 | 75.51 | 0.00 | 76.88 | 0.04 | 99.37 |
| R133b | 0.45 | 0.00 | 0.46 | 0.00 | 0.60 |
| R133 | 0.00 | 0.00 | 0.00 | 0.00 | 0.00 |
| R123a | 0.00 | 0.00 | 0.00 | 0.00 | 0.00 |
| R152a | 0.00 | 0.00 | 0.00 | 0.00 | 0.00 |
| R142 | 0.00 | 0.00 | 0.00 | 0.00 | 0.00 |
| R160 | 0.00 | 0.00 | 0.00 | 0.00 | 0.00 |
| R1113 | 0.00 | 0.00 | 0.00 | 0.00 | 0.00 |
| R1123 | 0.40 | 21.82 | 0.01 | 0.05 | 0.00 |
| R1132Z | 22.23 | 7.24 | 22.50 | 99.24 | 0.03 |
| R1132E | 1.06 | 51.15 | 0.15 | 0.66 | 0.00 |
| R1132a | 0.35 | 19.79 | 0.00 | 0.00 | 0.00 |

The reaction product from the second reactor in the integrated process is sent to the first distillation column to remove HFO-1132a and HFO-1123 from the product stream. The first distillation column may have 30 stages. The top stream and bottom stream compositions from the first distillation column are shown in Table 22.

The top stream from the first distillation column is recycled (e.g., to be optionally further hydrogenated in a separate reactor) and the bottom stream from the first distillation column is sent to the third distillation column to separate HFO-1132Z/E from unreacted HFC-143. The third distillation column may have 30 stages. The top stream and bottom stream compositions from the third distillation column is shown in Table 22. The top stream comprises of 1132 Z/E with a purity greater than 99.9%, which may be sent to a Step (iii) reactor for HFO-1132Z isomerization to HFO-1132E. The bottom stream comprises of HFC-143 with a purity greater than 99.3%, which is sent back to the Step (ii) liquid phase reactor for defluorination reaction.

### Example 15

### Formation of HFO-1132E

The HFO-1132Z/E isomers produced in Examples above are further reacted with a catalyst to produce HFO-1132E using Step (iii), as described above.

### Example 16

### Isomerization of HFO-1132(Z) to HFO-1132(E)

Table 39 shows the feed composition used for the isomerization reaction in the order each component appears in the gas chromatogram.

TABLE 39
Isomerization Reaction Feed Component Concentrations

| Component name | GC-FID area % | Conc. (ppm) |
|---|---|---|
| HFC-143a | <0.0000 | <1 |
| HFO-1123 | 0.0005 | 5 |
| HFO-1132(E) | 0.0079 | 79 |
| HFC-152a | 0.4218 | 4218 |
| HFO-1132(Z) | 98.8897 | 988897 |
| HFC-143 | 0.4830 | 4830 |
| Other | 0.1970 | 1970 |

After running the isomerization reaction using the above feed, different concentrations of CTFE co-feed as the catalyst, and nitrogen gas a diluent, a product composition as shown below was obtained. Reactions were performed at 490 °C and 30 psig in an empty ¾″ Inconel tube with about 14-25 seconds residence time. Reactions were performed in the presence of different CTFE weight percentages ranging from 4 wt. % to 13 wt. %. The 1132Z conversion and selectivity toward 1132E in the isomerization reaction is a function of CTFE concentration.

Table 40 below shows the chemical composition of the crude product collected during parametric studies (different CTFE concentrations and residence time) in the order they appeared in the gas chromatogram. Note that this material was not originated from single run conditions and the presented compositions should not be used to assess the isomerization reaction efficiency. However, the presented example shows how certain impurities impacted the quality of final 1132E product. For example, some impurities such as 1132a and 1141 are separable from 1132E while other impurities such as 143a and 1123 are not separable.

TABLE 40
Crude Product Composition Following Isomerization

| Component name | GC-FID area % | Conc. (ppm) |
|---|---|---|
| HFO-1132a | 0.0052 | 52 |
| HFC-143a | 0.0022 | 22 |
| HFO-1141 | 0.0243 | 243 |
| HFO-1123 | 0.0005 | 5 |
| HFO-1132(E) | 20.4650 | 204650 |
| HFC-152a | 1.4295 | 14295 |
| HFO-1132(Z) | 65.2216 | 652216 |
| HCFO-1113 | 7.8746 | 78746 |
| HFC-143 | 0.8753 | 8753 |
| Other | 4.0975 | 40975 |

Distillation of the crude product in Table 40 was performed to form a purified product composition. After distillation of the above crude product composition, a product with the below purity profiles was obtained, as shown in Table 41.

TABLE 41
Purified Product Composition Following Distillation

| Component name | GC-FID area % | Conc. (ppm) |
|---|---|---|
| HFC-32 | 0.0010 | 10 |
| HFC-143a | 0.0089 | 89 |
| HFO-1141 | 0.0005 | 5 |
| HFO-1123 | 0.0095 | 95 |
| HFO-1132(E) | 99.9746 | 999746 |
| HFC-152a | 0.0000 | 0 |
| HFO-1132(Z) | 0.0023 | 23 |
| HCFO-1113 | 0.0018 | 18 |
| HFC-143 | 0.0002 | 2 |
| Other | 0.0013 | 13 |

### Example 17

### Isomerization of HFO-1132(Z) to HFO-1132(E)

Table 42 shows the feed composition used for isomerization reaction in the order they appear in the gas chromatogram.

TABLE 42
Isomerization Reaction Feed Component Concentrations

| Component name | GC-FID area % | Conc. (ppm) |
|---|---|---|
| HFC-143a | <0.0000 | <1 |
| HFO-1123 | 0.0028 | 28 |
| HFO-1132(E) | 0.0851 | 851 |
| HFC-152a | 0.0214 | 214 |
| HFO-1132(Z) | 99.3757 | 993757 |
| HCFO-1113 (CTFE) | 0.0008 | 8 |
| HFC-143 | 0.3449 | 3449 |
| Other | 0.1693 | 1693 |

After running the isomerization reaction using the above feed, different concentrations of CTFE co-feed as the catalyst, and 1234yf gas a diluent, a product composition as shown below was obtained. Reactions were performed at 490 °C and 5-30 psig in an empty ¾″ Inconel tube with about 6-16 seconds residence time. Reactions were performed in the presence of different CTFE weight percentages ranging from 4 wt. % to 13 wt. %. The 1132Z conversion and selectivity toward 1132E in the isomerization reaction is a function of CTFE concentration; this relationship should be discussed either in another example or another case.

Table 43 below shows the chemical composition of the crude product collected during parametric studies (different CTFE concentrations and residence time) in the order they appear in the gas chromatogram. Note that this material was not originated from single run conditions and the presented compositions should not be used to assess the isomerization reaction efficiency. However, the presented example shows how certain impurities impacted the quality of the final 1132E product. For example, some impurities such as 1234yf, 1132a and 1141 are separable from 1132E while other impurities such as 143a and 1123 are not separable.

TABLE 43
Crude Product Composition Following Isomerization

| Component name | GC-FID area % | Conc. (ppm) |
|---|---|---|
| HFO-1132a | 0.0219 | 219 |
| HFC-143a | 0.0087 | 87 |
| HFO-1141 | 0.0625 | 625 |
| HFO-1123 | 0.0000 | <1 |
| HFO-1132(E) | 9.1708 | 91708 |
| HFO-1234yf | 49.4758 | 494758 |
| HFC-152a | 0.0123 | 123 |
| HFO-1132(Z) | 30.7896 | 30789 |
| HCFO-1113 | 8.8440 | 88440 |
| HFC-143 | 0.3848 | 3848 |
| Other | 1.2296 | 12296 |

TABLE 44
Crude Product Composition of Table 38 Excluding 1234yf

| Component name | GC-FID area % | Conc. (ppm) |
|---|---|---|
| HFO-1132a | 0.0433 | 433 |
| HFC-143a | 0.0172 | 172 |
| HFO-1141 | 0.1237 | 1237 |
| HFO-1123 | 0.0000 | <1 |
| HFO-1132(E) | 18.1513 | 181513 |
| HFC-152a | 0.0243 | 243 |
| HFO-1132(Z) | 60.9403 | 609403 |
| HCFO-1113 | 17.5045 | 175045 |
| HFC-143 | 0.7616 | 7616 |
| Other | 2.4337 | 24337 |

After distillation of the above crude product composition (Table 39), a purified product composition was obtained, as shown in Table 45.

TABLE 45
Purified Product Composition Following Distillation

| Component name | GC-FID area % | Conc. (ppm) |
|---|---|---|
| HFC-32 | 0.0042 | 42 |
| HFC-143a | 0.0054 | 54 |
| HFO-1141 | 0.0500 | 500 |
| HFO-1123 | 0.2450 | 2450 |
| HFO-1132(E) | 99.6541 | 996541 |
| HFC-152a | 0.0000 | <1 |
| HFO-1234yf | 0.0095 | 95 |
| HFO-1132(Z) | 0.0075 | 75 |
| HCFO-1113 | 0.0075 | 75 |
| HFC-143 | 0.0009 | 9 |
| Other | 0.0159 | 159 |

### Example 18

### Forming a Purified HfO-1132E Composition

Example 16 is repeated with various feed compositions in which the combined concentration of HFO-1123 and HFC-143a impurities in HFO-1132Z feed was varied. Table 41 shows how the combined concentration of HFO-1123 and HFC-143a impurities affect the purity of HFO-1132(E) product. In order to reach a minimum purity of 99.5%, the combined concentration of HFO-1123 and HFC-143a impurities should be less than 1500 ppm, preferably less than 1000 ppm, more preferably less than 200 ppm, and most preferably less than 25 ppm.

TABLE 46
HFO-1132(E) purity as a function of feed impurity concentration

| Combined concentration of HFO-1123 | |
|---|---|
| and HFC-143a impurities of the HFO- | Purity of HFO-1132(E) |
| 1132Z feed (ppm) | % |
| 2000 | 99.3452 |
| 1500 | 99.5050 |
| 1000 | 99.6648 |
| 200 | 99.9214 |
| 25 | 99.9767 |

### Example 19

Dehydrofluorination Reaction of Step (ii) with Isopropanol

49.8 grams of 85% KOH pellets were dissolved in 198.3 grams of isopropanol (IPA) in a 450 ml SS autoclave equipped with an agitator, a gas entrainer and internal thermocouple. To this solution, 39.2 grams HFC-143 (98.9% pure) were added to the autoclave and sealed. The autoclave was heated to 100 °C, stirring at 630 rpm, and maintained at 100 °C for 4.5-5 hours. The maximum pressure at 100 °C was 85 psig. After 4.5-5 hours, the heating was stopped and stirring was slowed to 280 rpm. The gaseous product was vented slowly into a 150 ml SS evacuated cylinder cooled with liquid N2. The internal temperature of the autoclave slowly cooled down to 70 °C with stirring during the product the collection period. 33.3 grams of organic material was recovered in the cylinder.

The organic product was analyzed using gas chromatography, results are shown in Table 42. It is assumed that the GC-FID area % are equal to weight %, and conversion and selectivity values were calculated using the equations shown above. Results are presented in Table 42.

TABLE 47
GC-FID area % of the recovered organic product

| Molecule | Product |
|---|---|
| R143 | 48.05 |
| R1132Z | 41.49 |
| R1132E | 3.02 |
| R1132a | 0.97 |
| Isopropanol | 1.09 |
| CHF2CH2OCH(CH3)2 | 4.18 |
| Others | 1.2 |

Conversion of R143 was 59.2%, 1132Z/E (combined) was 83.8%, 1132a selectivity was 1.8%.

A total of 3 runs (including Example XX) was performed as summarized in Table 48 and Table 49.

TABLE 48

| | | | mole | | | |
|---|---|---|---|---|---|---|
| | | | ratio | | | |
| | | | of | Selec. | Selec. | |
| | R143 | Organic | KOH | to | to | |
| | input, | recovered, | to | 1132 | 1132a | Conv. |
| Run # | grams | grams | Org | % | % | % |
| 1 | 45.1 | 43.5 | 1.2 | 87.6 | 8.9 | 20.7 |
| 2 | 51.8 | 41.6 | 1.1 | 64.1 | 3.9 | 45.8 |
| 3 | 39.2 | 33.3 | 1.6 | 83.8 | 1.8 | 59.2 |

TABLE 49

| | Run # | | |
|---|---|---|---|
| Molecule | 1 | 2 | 3 |
| R143 % | 82.26 | 67.49 | 48.05 |
| 1132Z % | 11.02 | 25.83 | 41.49 |
| 1132E % | 0.83 | 2 | 3.02 |
| 1132a % | 1.2 | 1.72 | 0.97 |
| IPA % | 3.29 | 2.06 | 1.09 |
| CHF2CH2OCH(CH3)2 % | 0.55 | 0.18 | 4.18 |
| Others % | 0.85 | 0.72 | 1.2 |

### Example 20

### Dehydrofluorination Reaction of Step (ii) Using Alternative Bases

Example 19 above is repeated with using the same reaction apparatus and conditions, but with sodium hydroxide (NaOH). Similar results are obtained.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### ASPECTS

Aspect 1 is a method for reducing impurities in a composition comprising 1,1,2-trifluoroethane (HFC-143), comprising: providing a first reactant composition comprising HFC-143 and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a); and reacting the first reactant composition with a base in a first liquid phase reactor to produce a first product composition comprising from 70 mol % to 99.8 mol % of 1,1,2-trifluoroethane (HFC-143), based on total moles of organic components of the first product composition.

Aspect 2 is the method of Aspect 1, wherein the providing the first reactant composition comprises: reacting 1,1,2-trichloro-1,2,2-trifluoroethane (CFC-113) with hydrogen in the presence of a first catalyst at a temperature of from 150 °C to 400 °C to produce the first reactant composition comprising at least 60 mol % and less than or equal to 80 mol % HFC-143, based on total moles of organic components of the first reactant composition; or reacting CTFE (CFO-1113) with hydrogen in the presence of a second catalyst at a temperature of from 75 °C to 400 °C to produce the first reactant composition comprising at least 60 mol % and less than or equal to 95 mol % HFC-143, based on total moles of organic components of the first reactant composition.

Aspect 3 is the method of either Aspect 1 or 2, wherein the base comprises at least one of KOH, NaOH, CaO, Ca(OH)₂, and combinations of the foregoing.

Aspect 4 is the method of any one of Aspects 1 to 3, wherein the reacting step is conducted at a temperature of from 80 °C to 130 °C.

Aspect 5 is the method of any one of Aspects 1 to 4, wherein the mole ratio of the base to the combined amount of HFC-143, HCFC-133b, HCFC-133, and HCFC-123a in the first reactant composition is from about 0.1 to about 1.5.

Aspect 6 is the method of any one of Aspects 1 to 5, wherein the mole ratio of the base to the combined amount of HCFC-133b, HCFC-133, and HCFC-123a in the first reactant composition is from about 0.5 to about 10.0.

Aspect 7 is the method of any one of Aspects 1 to 6, wherein the weight percentage of the base is from about 5 wt. % to about 50 wt. %, based on total weight of the base.

Aspect 8 is the method of any one of Aspects 1 to 7, wherein the reacting step achieves at least one of: a conversion of HCFC-123a of at least about 80%; a conversion of HCFC-133 and HCFC-133b of at least about 20%; and a conversion of HFC-143 of less than about 25%.

Aspect 9 is the method of any one of Aspects 1 to 8, wherein the reacting step achieves at least one of: a selectivity towards CTFE of at least about 5%; a selectivity towards HFC-1123 of at least about 10%; and a selectivity towards HFO-1132 of at least about 20%.

Aspect 10 is the method of any one of Aspects 1 to 9, wherein the first product composition comprises: 70 mol % to 99.8 mol % 1,1,2-trifluoroethane (HFC-143); 0.1 mol % to 15 mol % 1-chloro-1,1,2-trifluoroethane (HCFC-133b); 0.1 mol % to 7 mol % 1-chloro-1,2,2-trifluoroethane (HCFC-133); and 0 mol % to 3 mol % 1,2-dichloro-1,1,2-trifluoroethane (HCFC-123a), based on the combined total moles of the HFC-143, HCFC-133b, HCFC-133, and HCFC-123a in the product composition.

Aspect 11 is the method of Aspect 10, wherein the first product composition comprises a total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) of at least 80 mol %, based on total moles of organic components of the first product composition.

Aspect 12 is the method of any one of Aspects 1 to 11, further comprising: distilling the first product composition to produce a first distilled composition comprising at least 90 mol % HFC-143.

Aspect 13 is the method of Aspect 12, further comprising: reacting the distilled composition with a second base in a second liquid phase reactor to produce a second product composition; wherein the second reacting step is conducted at a temperature higher than a temperature of the first reacting step.

Aspect 14 is the method of Aspect 13, wherein the second base comprises at least one of KOH, NaOH, CaO, Ca(OH)₂, and combinations of the foregoing.

Aspect 15 is the method of Aspects 13 or 14, wherein the second reacting step is conducted at a temperature of from 100 °C to 150 °C.

Aspect 16 is the method of any one of Aspects 13 to 15, wherein the mole ratio of the second base to the combined amount of HFC-143, HCFC-133b, HCFC-133, and HCFC-123a in the distilled composition is from about 0.1 to about 2.0.

Aspect 17 is the method of any one of Aspects 13 to 16, wherein the weight percentage of the second base is from about 5 wt. % to about 50 wt. %, based on the total weight of the second base.

Aspect 18 is the method of any one of Aspects 13 to 17, wherein the second reacting step achieves a conversion of HFC-143 of at least about 25%.

Aspect 19 is the method of any one of Aspects 13 to 18, wherein the second reacting step achieves a selectivity towards HFO-1132 Z and/or E of at least about 35%.

Aspect 20 is the method of any one of Aspects 13 to 19, wherein the second product composition comprises: 20 mol % to 30 mol % of HFO-1132 Z and/or E; 70 mol % to 75 mol % of HFC-143; and 0 mol % to 5 mol % of a total amount of HCFC-133 isomers, HFC-152a, CTFE, HFO-1123, and HFO-1132a, based on total moles of organic components of the second product composition.

Aspect 21 is the method of any one of Aspects 13 to 20, wherein the second product composition comprises: 20 mol % to 45 mol % HFO-1132 Z and/or E; 50 mol % to 75 mol % HFC-143; and less than 5 mol % of a combined amount of HCFC-133 isomers, HFC-152a, CTFE, HFO-1123, and HFO-1132a, based on total moles of organic components of the second product composition.

Aspect 22 is the method of any one of Aspects 13 to 21, further comprising: distilling the second product composition to produce a second distilled composition comprising from 20 mol % to 50 mol % HFO-1132Z and from 50 mol % to 80 mol % HFC-143, based on total moles of organic components of the second distilled composition.

Aspect 23 is the method of Aspect 22, further comprising: distilling the second distilled composition to produce a third distilled composition comprising at least 99 mol % of HFO-1132Z, based on total moles of organic components of the third distilled composition.

Aspect 24 is the method of Aspect 23, wherein the third distilling step produces a recycle composition comprising at least 99 mol % of HFC-143, based on total moles of organic components of the recycle composition.

Aspect 25 is the method of any one of Aspects 1 to 12, wherein the reacting the first reactant composition in the first liquid phase reactor further comprises adding a phase transfer agent to the first liquid phase reactor.

Aspect 26 is the method of Aspect 25, wherein the phase transfer agent comprises at least one of C₂₅H₅₄ClN, quaternary ammonium salts, quaternary phosphonium salts, quaternary arsenium salts, sulfonium salts, crown ethers and similar molecules, and combinations of the foregoing.

Aspect 27 is the method of any one of Aspects 13 to 24, wherein the reacting the distilled composition in the second liquid phase reactor further comprises adding a phase transfer agent to the first liquid phase reactor.

Aspect 28 is the method of Aspect 27, wherein the phase transfer agent comprises at least one of C₂₅H₅₄ClN, quaternary ammonium salts, quaternary phosphonium salts, quaternary arsenium salts, sulfonium salts, crown ethers and similar molecules, and combinations of the foregoing.

Aspect 29 is a composition comprising: cis-1,2-difluoroethylene (HFO-1132(Z)) in an amount of at least 97 wt. %; 1,1,1-trifluoroethane (HFC-143a) and trifluoroethylene (HFO-1123) together present in a combined amount of greater than 0 ppm and less than 2000 ppm; 1,1,2-trifluoroethane (HFC-143) in an amount greater than 0.01 wt. % and less than 0.75 wt. %; and 1,1-difluoroethane (HFC-152a) in an amount greater than 0.01 wt. % and less than 0.75 wt. %, wherein all amounts are based on a total weight of organic components of the composition.

Aspect 30 is the composition of Aspect 29, wherein the HFC-143a and HFO-1123 together are present in a combined amount of greater than 0 ppm and less than 500 ppm, based on a total weight of organic components of the composition.

Aspect 31 is the composition of Aspect 29, wherein the HFC-143a and HFO-1123 together are present in a combined amount greater than 0 ppm and less than 100 ppm, based on a total weight of organic components of the composition.

Aspect 32 is the composition of Aspect 29, wherein the HFO-1132(Z) is present in an amount of at least 99 wt. %, based on a total weight of organic components of the composition.

Aspect 33 is the composition of Aspect 29, wherein the HFC-143 is present in an amount greater than 0.01 wt. % and less than 0.50 wt. %, based on a total weight of organic components of the composition.

Aspect 34 is the composition of Aspect 29, wherein the HFC-152a is present in an amount greater than 0.01 wt. % and less than 0.50 wt. %, based on a total weight of organic components of the composition.

Aspect 35 is a method for producing HFO-1132(E), comprising: reacting a reactant composition at a temperature of 490 °C, a pressure of 5 psig to 30 psig, and a residence time of 6 seconds to 25 seconds, the reactant composition comprising: cis-1,2-difluoroethylene (HFO-1132(Z)) in an amount of at least 97 wt. %; 1,1,1-trifluoroethane (HFC-143a) and trifluoroethylene (HFO-1123) together present in a combined amount of greater than 0 ppm and less than 2000 ppm; 1,1,2-trifluoroethane (HFC-143) in an amount greater than 0.01 wt. % and less than 0.75 wt. %; and 1,1-difluoroethane (HFC-152a) in an amount greater than 0.01 wt. % and less than 0.75 wt. %, wherein all amounts are based on a total weight of organic components of the reactant composition, to produce a crude product composition.

Aspect 36 is the method of Aspect 35, wherein the crude product composition comprises: HFO-1132(E) in an amount of at least 10 wt. %; HFO-1132(Z) in an amount from 25 wt. % to 70 wt. %; HFC-143a and HFO-1123 together present in a combined amount of greater than 0 ppm and less than 2000 ppm; HFC-143 in an amount greater than 0.01 wt. % and less than 1.00 wt. %; and HFC-152a in an amount greater than 0.01 wt. % and less than 1.50 wt. %, wherein all amounts are based on a total weight of organic components of the crude product composition.

Aspect 37 is the method of Aspect 36, further comprising conducting a distillation of the crude product composition to form a purified product composition; wherein the purified product composition comprises greater than 97 wt. % of HFO-1132(E), based on a total weight of organic components of the purified product composition.

Aspect 38 is the method of Aspect 35, wherein the HFC-143a and HFO-1123 together are present in the reactant composition in a combined amount greater than 0 ppm and less than 500 ppm, based on a total weight of organic components of the reactant composition.

Aspect 39 is the method of Aspect 35, wherein the HFC-143a and HFO-1123 together are present in the reactant composition in a combined amount greater than 0 ppm and less than 100 ppm, based on a total weight of organic components of the reactant composition.

Aspect 40 is the method of Aspect 35, wherein the HFO-1132(Z) is present in the reactant composition in an amount of at least 99 wt. %, based on a total weight of organic components of the reactant composition.

Aspect 41 is the method of Aspect 35, wherein the HFC-143 is present in the reactant composition in an amount greater than 0.01 wt. % and less than 0.50 wt. %, based on a total weight of organic components of the reactant composition.

Aspect 42 is the method of Aspect 35, wherein the HFC-152a is present in the reactant composition in an amount of greater than 0.01 wt. % and less than 0.50 wt. %, based on a total weight of organic components of the reactant composition.

Aspect 43 is the method of Aspect 35, wherein the crude product composition comprises HFO-1132(E) in an amount of at least 18 wt. %, based on a total weight of organic components of the crude product composition.

Aspect 44 is the method of Aspect 35, wherein the crude product composition comprises HFO-1132(Z) in an amount from 30 wt. % to 66 wt. %, based on a total weight of organic components of the crude product composition.

Aspect 45 is the method of Aspect 35, wherein the crude product composition comprises: HFC-143a and HFO-1123 together present in a combined amount of greater than 0 ppm and less than 700 ppm; HFC-143 in an amount greater than 0.01 wt. % and less than 0.88 wt. %; and HFC-152a in an amount greater than 0.01 wt. % and less than 1.00 wt. %, wherein all amounts are based on a total weight of organic components of the crude product composition.

Aspect 46 is the method of Aspect 35, wherein the reacting step further comprises at least one of: reacting the reactant composition in the presence of a diluent, wherein the diluent is nitrogen gas; and reacting the reactant composition in the presence of a catalyst, wherein the catalyst is chlorotrifluoroethylene (CTFE).

Aspect 47 is a method of producing HFO-1132(Z) comprising: providing a reactant composition comprising HFC-143; and reacting the reactant composition with a base in a liquid phase reactor to produce a product composition comprising HFO-1132(Z).

Aspect 48 is the method of Aspect 47, wherein the base comprises at least one of KOH, NaOH, CaO, Ca(OH)2, and combinations of the foregoing.

Aspect 49 is the method of Aspect 47 or Aspect 48, wherein the reacting step is conducted at a temperature from about 100 °C to about 150 °C.

Aspect 50 is the method of any one of Aspects 47 to 49, wherein the reactant composition further comprises at least about 0 mol. % of HCFC-133b, HCFC-133, and HCFC-123a, and the mole ratio of the base to the combined amount of HFC-143, HCFC-133b, HCFC-133, and HCFC-123a, in the reactant composition is from about 0.1 to about 2.0.

Aspect 51 is the method of any one of Aspects 47 to 50, wherein the reacting step achieves a conversion of HFC-143 of at least about 25%.

Aspect 52 is the method of any one of Aspects 47 to 51, wherein the reacting step achieves a selectivity towards HFO-1132 Z and or E of at least about 35%.

Aspect 53 is the method of any one of Aspects 47 to 52, wherein the product composition comprises: about 20 mol % to about 30 mol % of HFO-1132Z and/or E; about 70 mol % to about 75 mol % of HFC-143; and about 0 mol % to about 5 mol % of a total amount of HCFC-133 isomers, HFC-152a, CTFE, HFO-1123, and HFO-1132a, based on total moles of organic components of the second product composition.

Aspect 54 is the method of any one of Aspects 47 to 53, further comprising: distilling the product composition to produce a first distilled composition and a first bottom composition, the first bottom composition comprising water and unreacted HFC-143.

Aspect 55 is the method of Aspect 54, further comprising: recycling the first bottom composition wherein at least a portion of the first bottom composition is provided to the liquid phase reactor.

Aspect 56 is the method of Aspect 54 or Aspect 55, further comprising: drying the first distilled composition to produce a dried distilled composition; distilling the dried distilled composition to create a second distilled composition and a second bottom composition, the second bottom composition comprising HFC-143 and at least about 15 mol. % HFO-1132Z based on the total moles of organic components of the second distilled composition; distilling the second bottom composition to produce a third distilled composition and a third bottom composition, the third distilled composition comprising at least about 99 mol % of HFO-1132Z based on the total moles of organic components of the third distilled composition, and the third bottom composition comprising HFC-143; distilling the third bottom composition to produce a fourth distilled composition comprising at least 80 mol % HFC-143; and recycling the fourth distilled composition to the liquid phase reactor.

Aspect 57 is cis-1,2-difluoroethylene (HFO-1132(Z)) produced according to the method of any one of Aspects 47 to 56.

What is claimed is:

1. A composition comprising:
cis-1,2-difluoroethylene (HFO-1132(Z)) in an amount of at least 97 wt. %;
1,1,1-trifluoroethane (HFC-143a) and trifluoroethylene (HFO-1123) together present in a combined amount of greater than 0 ppm and less than 2000 ppm;
1,1,2-trifluoroethane (HFC-143) in an amount greater than 0.01 wt. % and less than 0.75 wt. %; and
1,1-difluoroethane (HFC-152a) in an amount greater than 0.01 wt. % and less than 0.75 wt. %, wherein all amounts are based on a total weight of organic components of the composition.

2. The composition of claim 1, wherein the HFC-143a and HFO-1123 together are present in a combined amount of greater than 0 ppm and less than 500 ppm, based on a total weight of organic components of the composition.

3. The composition of claim 1, wherein the HFC-143a and HFO-1123 together are present in a combined amount greater than 0 ppm and less than 100 ppm, based on a total weight of organic components of the composition.

4. The composition of claim 1, wherein the HFO-1132(Z) is present in an amount of at least 99 wt. %, based on a total weight of organic components of the composition.

5. The composition of claim 1, wherein the HFC-143 is present in an amount greater than 0.01 wt. % and less than 0.50 wt. %, based on a total weight of organic components of the composition.

6. The composition of claim 1, wherein the HFC-152a is present in an amount greater than 0.01 wt. % and less than 0.50 wt. %, based on a total weight of organic components of the composition.

7. A method for producing cis-1,2-difluoroethylene (HFO-1132(E)) comprising reacting the composition of claim 1 to produce HFO-1123 (E).

8. A method of producing cis-1,2-difluoroethylene (HFO-1132(Z)) comprising:
providing a reactant composition comprising HFC-143; and
reacting the reactant composition with a base in a liquid phase reactor to produce a product composition comprising HFO-1132(Z).

9. The method of claim 8, wherein the base comprises at least one of KOH, NaOH, CaO, Ca(OH)2, and combinations of the foregoing.

10. The method of claim 8, wherein the reacting step is conducted at a temperature from about 100 °C to about 150 °C.

11. The method of claim 8, wherein the reactant composition further comprises at least about 0 mol. % of HCFC-133b, HCFC-133, and HCFC-123a, and the mole ratio of the base to the combined amount of HFC-143, HCFC-133b, HCFC-133, and HCFC-123a, in the reactant composition is from about 0.1 to about 2.0.

12. The method of claim 8, wherein the reacting step achieves a conversion of HFC-143 of at least about 25%.

13. The method of claim 8, wherein the reacting step achieves a selectivity towards HFO-1132(Z) and or E of at least about 35%.

14. The method of claim 8, wherein the product composition comprises:
about 20 mol % to about 30 mol % of HFO-1132(Z) and/or E;
about 70 mol % to about 75 mol % of HFC-143; and
about 0 mol % to about 5 mol % of a total amount of HCFC-133 isomers, HFC-152a, CTFE, HFO-1123, and HFO-1132a, based on total moles of organic components of the second product composition.

15. The method of claim 8, further comprising:
distilling the product composition to produce a first distilled composition and a first bottom composition, the first bottom composition comprising water and unreacted HFC-143.

16. The method of claim 15, further comprising:
recycling the first bottom composition wherein at least a portion of the first bottom composition is provided to the liquid phase reactor.

17. The method of claim 15, further comprising:
drying the first distilled composition to produce a dried distilled composition;
distilling the dried distilled composition to create a second distilled composition and a second bottom composition, the second bottom composition comprising HFC-143 and at least about 15 mol. % HFO-1132(Z) based on the total moles of organic components of the second distilled composition; and
distilling the second bottom composition to produce a third distilled composition and a third bottom composition, the third distilled composition comprising at least about 99 mol % of HFO-1132(Z) based on the total moles of organic components of the third distilled composition, and the third bottom composition comprising HFC-143.

18. The method of claim 17, further comprising distilling the third bottom composition to produce a fourth distilled composition comprising at least 80 mol % HFC-143; and
recycling the fourth distilled composition to the liquid phase reactor.

19. The method of claim of claim 8 further comprising reacting the product composition to produce a second product composition comprising HFO-1132(E).

20. A product composition comprising cis-1,2-difluoroethylene (HFO-1132(E)) produced according to the method of claim 19.
